(19) United States
(12) Patent Application Publication
(10) Pub. No.: US 2006/0100154 A1
(43) Pub. Date: May 11, 2006

(54) Long-acting gonadotropin-releasing hormone analogs and methods of use thereof

(75) Inventors: Yitzhak Koch, Rehovot, IL (US); Matityahu Fridkin, Rehovot (IL); Shai Rahimipour, Netanya (IL)

Correspondence Address: HESLIN ROTHENBERG FARLEY & MESITI PC, 5 COLUMBIA CIRCLE, ALBANY, NY, 12203, US

(73) Assignee: YEDA RESEARCH AND DEVELOPMENT CO, Rehovot, IL (US)

(21) Appl. No.: 10/530,100

(22) PCT Filed:
(86) PCT No.: PCT/IL02/00801

(51) Int. Cl. A61K 38/09

(52) U.S. Cl. 514/15; 514/16

(57) ABSTRACT

The present invention relates to the design, synthesis and biological evaluation of potent long-acting gonadotropin-releasing hormone (GnRH) analogs including agonists and antagonists comprising a GnRH peptide conjugated to emodic acid or an emodic acid derivative. These long acting analogs bind to GnRH receptors with high affinity and are devoid of any toxicity or antiproliferative effects. The present invention further relates to therapeutic uses of these GnRII analogs as contraceptives, in controlling fertility and in treating and/or preventing sex-hormone dependent diseases or conditions.

### FIELD OF INVENTION

The present invention relates to the design, synthesis and biological evaluation of potent long-acting gonadotropin-releasing hormone (GnRH) analogs, and to their therapeutic use in fertility regulation or as contraceptives, and in treating and/or preventing sex hormone-related diseases or conditions.

### BACKGROUND OF THE INVENTION

Gonadotropin-releasing hormone (GnRH; pGlu-His-Trp-Ser-Tyr-Gly-Leu-Arg-Pro-Gly-NH₂) is a key integrator between the neural and the endocrine systems and plays a pivotal role in the regulation of the reproductive system. This neurohormone is synthesized in hypothalamic neurosecretory cells and is released in a pulsatile pattern into the hypothalamo-hypophyseal portal circulation. This pattern of GnRH secretion provokes the release of the gonadotropins, luteinizing hormone (LH) and follicle-stimulating hormone (FSH), from the anterior pituitary, which, in turn, stimulate gonadal steroidogenesis and gametogenesis^{1,2}. Chronic administration of GnRH or its super active agonists results in down-regulation of GnRH receptors and desensitization of the pituitary gonadotrophs and thus causes the suppression of gonadotropin secretion^{3,4}. Synthetic GnRH analogs, agonists as well as antagonists, have attracted remarkable interest because of their potential applications for the treatment of reproductive diseases, such as prostate and breast cancer, and their possible use as contraceptives^{5,6}. The mechanism of action of GnRH analogs in these diseases is believed to be at least partly related to gonadal steroids deprivation, which results from down-regulation and desensitization of the pituitary gonadotrophs. In cancer therapy however, GnRH analogs have been demonstrated to exert direct inhibitory effects on the growth of cancer cells through GnRH receptors that are present in prostate, breast and ovarian cancer^{5,7}.

The relatively short half-life time of GnRH in the general circulation (2-4 min)⁵ is advantageous for the establishment of a pulsatile secretion pattern. However, potent agonists or antagonists having a prolonged bioactivity are certainly needed in the clinic for the induction of desensitization or contraception. Since the discovery of GnRH, more than 3000 analogs of the peptide have been synthesized and evaluated for their bioactivity. Most of the super agonists usually incorporate a D-amino acid, substituting for Gly in position 6, and an N-ethylamide instead of the terminal Gly-NH₂ in position 10. These chemical modifications enhance the bioactive β-turn conformation of GnRH at the Gly⁶-Leu⁷ bond and decrease the susceptibility of the peptide to proteolytic degradation^{8,9}. In addition, increasing the hydrophobicity of the peptide, by incorporation of appropriate amino acid residues, usually results in increased biological potency, probably due to a decrease in the rate of clearance from the general circulation and an increase in its apparent binding constant to GnRH receptors^{10,11}.

Conjugation of bulky moieties, such as tetramethylrhodamine, to the ε-amino group of [D-Lys⁶]GnRH do not significantly affect the bioactivity of GnRH analogs¹². Thus, in an attempt to produce effective targeted chemotherapy against cancer, several chemotherapeutic agents have been covalently attached to the ε-amino group of [D-Lys⁶]GnRH¹³⁻¹⁵. For example, attachment of the cytotoxic compound 2-(hydroxymethyl)anthraquinone hemiglutarate¹⁶ (FIG. 1) to [D-Lys⁶]GnRH has generated a powerful agonist, T-98, which bound to GnRH receptors on human breast cancer cells with a binding affinity similar to that of [D-Lys⁶]GnRH. This analog, however, exhibited a five-fold higher biological activity than the parent peptide and inhibited the growth of human breast and prostate cancer¹⁴.

Long acting antagonists of GnRH produced by modification of positions 5 or 6 in the peptide sequence are disclosed for example in U.S. Pat. No. 6,214,798, and references therein. The present inventors have published their initial findings concerning the longer duration of action of a particular GnRH agonist [D-Lys⁶]GnRH conjugated to emodic acid^{14a}

Development of new potent GnRH agonists and antagonists is of a major interest to the medical community, largely because of their clinical application in modulating the reproductive system as well as in therapy of cancer and various other sex-hormone related conditions. Thus, there is an urgent need in the art for potent GnRH agonists and antagonists that do not exhibit any toxic effects, and that can be safely administered as contraceptives, and as therapeutic agents in the treatment of sex-hormone related conditions and diseases.

### SUMMARY OF THE INVENTION

The present invention relates to the design, synthesis and biological evaluation of potent long-acting gonadotropin-releasing hormone (GnRH) agonists or antagonists, comprising GnRH analogs conjugated to emodic acid or an emodic acid derivative. These agonists and antagonists have prolonged duration of action and are thus useful in the control of fertility or as a contraceptive, and in treating and/or preventing sex-hormone related diseases or conditions.

According to a currently preferred embodiment, the present invention relates to the design, synthesis and biological evaluation of the potent long-acting GnRH agonist, [D-Lys⁶(Emo)]GnRH. [D-Lys⁶(Emo)]GnRH binds GnRH receptors with high affinity to induce LH release, is devoid of toxicity, and is thus useful in the control of fertility or as a contraceptive, and in treating and/or preventing sex-hormone related diseases or conditions.

According to another currently preferred embodiment, the present invention relates to the design, synthesis and biological evaluation of the potent long-acting GnRH antagonist, [D-Pyr¹, D-Phe², D-Trp³, D-Lys⁶(Emo)]GnRH (denoted herein as [D-Lys⁶(Emo)]GnRH-Antg). [D-Lys⁶(Emo))GnRH-Antg binds GnRH receptors to prevent LH release, is devoid of toxicity, and is thus useful in the control of fertility or as a contraceptive, and in treating and/or preventing sex-hormone related diseases or conditions.

Additional GnRH agonists according to the invention are represented by [SEQ ID NO:1] peptides of the general formula:
Pyr-His-Trp-Y-Tyr-X-Leu-Arg-Pro-Z
wherein X is selected from Ser(Emo), D-Ser(Emo), Lys(Emo), D-Lys(Emo), D-Dab(Emo), D-Orn(Emo), D-hSer(Emo); Y is selected from D-Lys(Emo), D-Dab(Emo), D-Orn(Emo), D-Ser(Emo), D-hSer(Emo); Z is selected from Gly, Ethylamine, D-Ala; Pyr denotes pyroglutamic acid, Dab denotes diaminobutyric acid; and pharmaceutically acceptable salts, amides, esters and hydrates thereof.

Additional GnRH antagonists according to the invention are represented by [SEQ ID NO:2] peptides of the general formula:
Ac-D-Nal-4-chloro-D-Phe-β(3-Pyridyl)-D-Ala-X—Y-Z-Leu-W-Pro-D-Ala
wherein, X is selected from Ser(Emo), hSer(Emo); Y is selected from Lys(Emo), Dab(Emo), Z is selected from D-Lys(Emo), D-Dab (Emo), D-Orn(Emo), D-Ser(Emo) D-hSer(Emo); W is selected from Lys(Emo), Dab(Emo), Orn(Emo), Ser(Emo), hSer(Emo); and pharmaceutically acceptable salts, esters, amides and hydrates thereof.

Additional GnRH antagonists according to the invention are represented by [SEQ ID NO:3] peptides of the general formula:
Ac-Nal-4-chloro-D-Phe-D-Pal-X-Tyr-Y-Leu-Arg-Pro-D-Ala
wherein X is selected from Ser(Emo), hSer(Emo); Y is selected from D-Cit(Emo), D-Lys(Emo), D-Dab(Emo), D-Orn(Emo), D-Ser(Emo) D-hSer(Emo); D-Nal denotes D-3-(2′-naphtyl)-alanine; D-Pal denotes 3-(3′-pyridyl)-alanine; and pharmaceutically acceptable salts, amides, esters and hydrates thereof.

In one embodiment, the present invention provides a method of controlling fertility in a subject, comprising the step of administering to the subject a gonadotropin releasing hormone (GnRH) agonist or antagonist peptide having the formula [D-Lys⁶(Emo)]GnRH, or a pharmaceutically acceptable salt or hydrate thereof, in an amount effective to control fertility in the subject In one embodiment, fertility is controlled to prevent conception in a female mammalian subject.

In yet another embodiment, the present invention provides a method of contraception in a subject, comprising the step of administering to the subject a gonadotropin releasing hormone (GnRH) agonist or antagonist peptide having the formula [D-Lys⁶(Emo)]GnRH, or a pharmaceutically acceptable salt or hydrate thereof.

Furthermore, in another embodiment, the present invention provides a method of treating a sex hormone-related disease or condition in a subject, the method comprising the step of administering to the subject a gonadotropin releasing hormone (GnRH) agonist or antagonist peptide having the formula [D-Lys⁶(Emo)]GnRH, or a pharmaceutically acceptable salt or hydrate thereof, in an amount effective to treat the disease or condition in said subject.

Furthermore, in another embodiment, the present invention provides a method of preventing a sex hormone-related disease or condition in a subject, the method comprising the step of administering to the subject a gonadotropin releasing hormone (GnRH) agonist or antagonist peptide having the formula [D-Lys⁶(Emo)]GnRH, or a pharmaceutically acceptable salt or hydrate thereof, in an amount effective to prevent the disease or condition in the subject.

Furthermore, in another embodiment, the present invention provides a method of promoting the release of LH or FSH in a subject, the method comprising the step of administering to the subject a gonadotropin releasing hormone (GnRH) agonist or antagonist peptide having the formula [D-Lys⁶(Emo)]GnRH, or a pharmaceutically acceptable salt or hydrate thereof, in an amount effective to promote the release of LH or FSH in the subject.

Furthermore, in another embodiment, the present invention provides a method of suppressing or preventing the release of LH or FSH in a subject, the method comprising the step of administering to the subject a long-acting gonadotropin releasing hormone (GnRH) agonist or antagonist peptide having the formula [D-Lys⁶(Emo)]GnRH, or a pharmaceutically acceptable salt or hydrate thereof, in an amount effective to prevent or suppress the release of LH or FSH in the subject.

In one embodiment, the subject is a mammal. In another embodiment, the subject is a human. In another embodiment the subject is a non-human mammal. In another embodiment the subject is a non-mammalian vertebrate. In another embodiment, the subject is a male subject. In another embodiment, the subject is a female subject.

In one embodiment, the sex hormone-related disease or condition is a cancer, for example prostate cancer, breast cancer, ovarian cancer, cervical cancer, a tumor of the pituitary, testicular cancer or uterine cancer. In another embodiment, the sex hormone-related disease or condition is a benign disease or condition, for example benign prostatic hyperplasia, precocious puberty, aberrant sexual behavior, late luteal phase dysphoric disorder premenstrual syndrome), fibroids, endometriosis, myoma, hirsutism, cyclic auditory dysfunction, porphyria, or polycystic ovarian syndrome.

In one embodiment, the methods of the present invention comprise administering a pharmaceutical preparation comprising the GnRH agonist peptide, and a pharmaceutically acceptable carrier. In another embodiment, the pharmaceutical preparation is orally administered in solid or liquid dosage form. In another embodiment, the pharmaceutical preparation is intravenously, intraarterially, subcutaneously, intradermally, intraperitoneally, intramuscularly, intranasally or intralesionally injected in liquid form. In another embodiment, the pharmaceutical preparation is administered as an intravaginal device or ring. In another embodiment, the pharmaceutical preparation is formulated as a topical formulation for topical application. In another embodiment, the pharmaceutical preparation is formulated as a pellet, a tablet, a capsule, a solution, a suspension, an emulsion, a gel, a cream, a suppository, an intra-vaginal ring, or a parenteral formulation. In a particular embodiment the pharmaceutical preparation is formulated as a depot for providing sustained release of the active ingredient.

### BRIEF DESCRIPTION OF THE DRAWINGS

The present invention will be understood and appreciated more fully from the following detailed description taken in conjunction with the appended drawings which depict:

FIG. 1. Chemical structure of quinonic moieties: (1) 1,3,8 trihydroxy-6-methyl-9,10-anthraquinone (Emodin); (2) 1,3,8-trihydroxy-6-oxy-9,10-anthraquinone (Emodic acid, Emo), (3) 2-hydroxymethyl anthraquinone-hemiglutarate (AntrQ); and (4) 2-β-alanyl-3-chloro-1,4-naphthoquinone (NQ).

FIG. 2. Solid phase synthesis of [D-Lys⁶(Emo)]GnRH.

FIG. 3. Displacement (%) of specific binding of ¹²⁵I[D-Lys⁶]GnRH from pituitary membranes of proestrous rats by increasing concentrations of unlabeled GnRH analogs: [D-Lys⁶(AntQ)]GnRH (◯); [D-Lys⁶(NQ)]GnRH (▴); [D-Lys⁶(Emo)]GnRH (♦); and [D-Lys⁶]GnRH (□).

FIG. 4. LH releasing potency of [D-Lys⁶]GnRH conjugates in primary cultures of rat pituitary cells containing the indicated concentrations of [D-Lys⁶]GnRH (□); [D-Lys⁶(AntrQ)]GnRH (◯); [D-Lys⁶(NQ)]GnRH (▴); or [D-Lys⁶(Emo)]GnRH (♦).

FIG. 5. Effect of a GnRH antagonist ([D-pyr¹, D-Phe², D-Trp^{3,6}]GnRH) on the induction of LH secretion from primary cultures of rat pituitary cells stimulated by [D-Lys⁶(Emo)]GnRH.

FIG. 6. Induction of LH release in rats by intraperitoneal administration of [D-Lys⁶(Emo)]GnRH (♦) or of the parent peptide [D-Lys⁶]GnRH (□). The Y-axis is presented as a logarithmic scale.

FIG. 7. Effects of long-term administration of GnRH analogs on the weight of testes and prostate glands. Adult male rats were injected daily for 7 days either with [D-Lys⁶(Emo)]GnRH (0.1 or 1 nmol/rat, gray), [D-Lys⁶]GnRH (1 nmol/rat, white), or PBS (control, black). Rats were sacrificed 24 h after the last injection, and testes (a) and prostate glands (b) were dissected and weighed.

FIG. 8. Binding of Emodic acid and GnRH analogs to human serum albumin (HSA).

FIG. 9. Phototoxicity of [D-Lys⁶(Emo)]GnRH and emodic acid to αT3-1 cells. Cells were incubated; with [D-Lys⁶(Emo)]GnRH (black) or emodic acid (white), washed and illuminated. Cell survival was determined by the XTT method. Values are expressed as % survival. 100% survival (gray) refers to the survival of cells in the control group that were incubated without any emodic acid derivatives.

FIG. 10. DNA cleavage in cells treated with [D-Lys⁶(Emo)]GnRH and emodic acid. αT3-1 cells were incubated; in darkness with 10 μM of the tested compounds, washed and illuminated. After 24 h of incubation DNA was isolated and analyzed by gel electrophoresis. Lanes 1, 3 and 5 represent DNA of cells treated with 1% DMSO in PBS, emodic acid or [D-Lys⁶(Emo)]GnRH, respectively, followed by illumination. Lane 2, 4 and 6 represent cells treated with 1% DMSO in PBS, emodic acid, or [D-Lys⁶(Emo)]GnRH, respectively, in darkness.

FIG. 11. Displacement of ¹²⁵I[D-Lys⁶]-GnRH from proestrous rat pituitary membrane receptors by increasing concentrations of unlabeled GnRH analogs.

FIG. 12. The effect of a GnRH antagonist conjugate on the inhibition of LH secretion from primary cultures of rat pituitary cells that were stimulated by GnRH. Cells were incubated with GnRH (1 nM) in the absence or presence of increasing concentrations of [D-Lys⁶]Antg or [D-Lys⁶(Emo)]Antg. Following the incubation period (4 h at 37° C.) media were collected and LH concentration was determined by RIA. Results are the mean±SEM of two independent experiments (4 wells/experimental group, each).

### DETAILED DESCRIPTION OF THE INVENTION

The present invention relates to the design, synthesis and biological evaluation of potent long-acting gonadotropin-releasing hormone (GnRH) agonists and antagonists, which comprise a GnRH analog conjugated to emodic acid or an emodic acid derivative.

According to the principles of the present invention a GnRH analog is conjugated to a molecule having the general formula: 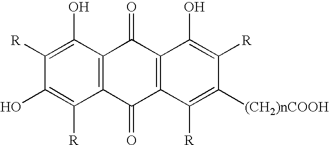
wherein n is an integer of 0-5 and R is independently at each occurrence selected from hydrogen, hydroxy, alkoxy, halogen, a straight chain, branched or cyclic alkyl group, lower alkenyl group, lower alkynyl group, carboxyl, carboxyalkyl, amino, aminoalkyl, diaminoalkyl, thio, thioalkyl, amido, alkylamido, dialkylamido or any other suitable substituent that yields a long acting non-toxic derivative of said GnRH analog.

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

A “hydroxy” group refers to an OH group. An “alkoxy” group refers to an —O-alkyl group wherein alkyl is as defined above. A “thio” group refers to an —SH group. A “thioalkyl” group refers to an —SR group wherein R is alkyl as defined above. An “amino” group refers to an —NH₂ group. An “alkylamino” group refers to an —NHR group wherein R is alkyl is as defined above. A “dialkylamino” group refers to an —NRR′ group wherein R and R′ are alkyl as defined above. An “amido” group refers to an —CONH₂ group. An “alkylamido” group refers to an —CONHR group wherein R is alkyl is as defined above. A “dialkylamido” group refers to an —CONRR′ group wherein R and R′ are alkyl as defined above. A “nitro” group refers to an NO2 group. A “carboxyl” group refers to a COOH group. A “carboxyalkyl” refers to a COOR group wherein R is an alkyl as defined above.

In one embodiment, the present invention relates to the design, synthesis and biological evaluation of a potent long-acting gonadotropin-releasing hormone (GnRH) agonist, ([D-Lys⁶(Emo)]GnRH) which binds GnRH receptors with high affinity to induce LH release, and which is devoid of any toxicity or antiproliferative effects. The present invention further relates to therapeutic uses of ([D-Lys⁶(Emo)]GnRH) as a contraceptive, and in treating and/or preventing sex-hormone dependent diseases or conditions.

In another embodiment, the present invention further relates to the design, synthesis and biological evaluation of a potent long-acting gonadotropin-releasing hormone (GnRH) antagonist, ([D-Lys⁶(Emo)]GnRH-Antg), which binds GnRH receptors with high affinity to prevent LE release, and which useful as a contraceptive, and in treating and/or preventing sex-hormone dependent diseases or conditions.

As defined herein a receptor agonist is a substance which binds receptors and activates them. A receptor antagonist is a substance which binds receptors and inactivates them. Assays to determine whether the compounds of the present invention are agonists or antagonists are well known to a person skilled in the art.

As discussed hereinabove, conjugation of bulky moieties to the ε-amino group of [D-Lys⁶]GnRH do not significantly affect the bioactivity of GnRH analogs. For example, incorporation of an anthraquinone moiety such as 2-hydroxymethyl anthraquinone hemiglutarate to [D-Lys⁶]GnRH, generates an agonist ([D-Lys⁶(AntrQ)]GnRH) with superior bioactivity to that of the parent peptide¹⁴, but which is cytotoxic to cells, and inhibits the growth of human breast and prostate cancer¹⁴.

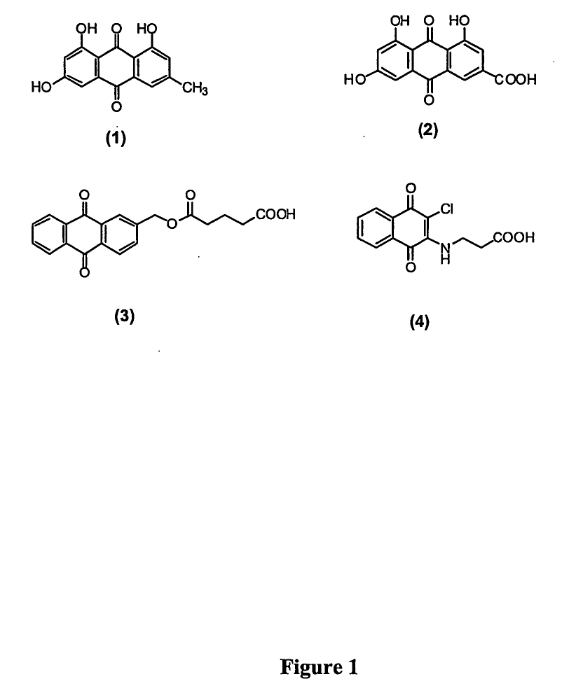

In an attempt to develop a GnRH agonist possessing similar or superior activity to [D-Lys⁶]GnRH, but which is devoid of any toxic or antiproliferative effects, Applicants have unexpectedly found that incorporation of the anthraquinone-based emodin moiety (1,3,8-trihydroxy-6-methyl-9,10-anthraquinone) to [D-Lys⁶]GnRH, generates a nontoxic, potent agonist ([D-Lys⁶(Emo)]GnRH), which binds with high affinity in-vitro and in-vivo to GnRH receptors to induce LH release. Emodin (FIG. 1) is a naturally occurring polyhydroxylated anthraquinone that is widely used for preparation of laxatives. Applicants have previously shown²⁴ that incorporation of emodic acid to [D-Lys⁶]GnRH diminished its ability to generate reactive radical species (ROS)²⁵.

Moreover, Applicants have unexpectedly found that ([D-Lys⁶(Emo)]GnRH) possesses long-term bioactivity^{14a}. Development of long acting GnRH analogs is of particular interest, since in the clinic GnRH analogs are frequently administered in slow-release depot preparations in order to desensitize the pituitary gland. Without wishing to be bound by any particular concept or mechanism of action, the long term bioactivity may be attributed at least in part, as demonstrated herein, to the ability of emodin to bind with high affinity to serum proteins such as human serum albumin (HSA)¹⁷, which may protect the peptide from proteolytic degradation.

Accordingly, the present invention provides a potent long-acting GnRH super-active agonist—([D-Lys⁶(Emo)]GnRH), or a long-acting GnRH antagonist,—[D-Lys6(Emo)]GnRH-Antg which is useful in the prevention and/or treatment of a variety of sex-hormone related conditions (specifically conditions involving cells that carry GnRH receptors), and as a contraceptive.

In one embodiment, [D-Lys⁶(Emo)]GnRH is useful as a contraceptive agent to control fertility in a subject, for example to prevent conception in a female subject. As described hereinabove, chronic administration of GnRH agonists results in down-regulation of GnRH receptors and desensitization of the pituitary gonadotrophs and thus causes the suppression of gonadotropin secretion^{3,4}. Thus, in one embodiment, [D-Lys6(Emo)]GnRH or [D-Lys6(Emo)]GnRH-Antg may be administered in an amount and dosage effective to suppress the release the gonadotropins LH and FSH, and thus may act as a contraceptive agent and prevent conception. In one embodiment, [D-Lys6(Emo)]GnRH or [D-Lys6(Emo)]GnRH-Antg is a potent contraceptive in males. In another embodiment, [D-Lys6(Emo)]GnRH or [D-Lys6(Emo)]GnRH-Antg is a potent contraceptive in females, i.e. is effective in preventing pregnancy in females. In another embodiment, [D-Lys6(Emo)]GnRH or [D-Lys6(Emo)]GnRH-Antg is a potent contraceptive in humans.

Thus, in one embodiment, the present invention provides a method of controlling fertility in a mammalian subject, comprising the step of administering to the subject a gonadotropin releasing hormone (GnRH) agonist or antagonist peptide having the formula [D-Lys⁶(Emo)]GnRH or [D-Lys⁶(Emo)]GnRH-Antg, or a pharmaceutically acceptable salt or hydrate thereof, in an amount effective to control fertility in the subject. In one embodiment, fertility is controlled to prevent conception in a female subject.

In yet another embodiment, the present invention provides a method of contraception in a mammalian subject, comprising the step of administering to the subject a gonadotropin releasing hormone (GnRH) agonist or antagonist peptide having the formula [D-Lys⁶(Emo)]GnRH or [D-Lys⁶(Emo)]GnRH-Antg or a pharmaceutically acceptable salt or hydrate thereof.

As defined herein, the term “analog” means any variant and includes both agonists and antagonists

As defined herein, the term “conception” means the onset of pregnancy, marked by the formation of a viable zygote and subsequent implantation of the blastocyst. As defined herein, the term “contraceptive” means an agent that diminishes the likelihood of or that prevents conception.

As used herein, the term “administering” refers to bringing a subject in contact with a pharmaceutical composition comprising a GnRH peptide of the present invention. As used herein, administration can be accomplished in vitro, i.e. in a test tube, or in vivo, i.e. in cells or tissues of living organisms, for example humans.

Furthermore, in accordance with another embodiment of the present invention, [D-Lys⁶(Emo)]GnRH or [D-Lys⁶(Emo)]GnRH-Antg is useful in the prevention and/or treatment of a sex-hormone related diseases or conditions. Thus, in one embodiment, the present invention provides a method of treating a sex hormone-dependent disease in a subject, the method comprising the step of administering to the subject [D-Lys⁶(Emo)]GnRH or [D-Lys⁶(Emo)]GnRH-Antg, or a pharmaceutically acceptable salt or hydrate thereof, in an amount effective to treat the disease or condition in the subject. In another embodiment, the present invention provides a method of preventing a sex hormone-dependent disease in a mammalian subject, the method comprising the step of administering to the subject [D-Lys⁶(Emo)]GnRH, or a pharmaceutically acceptable salt or hydrate thereof, in an amount effective to prevent the disease or condition in the subject.

As defined herein, the term “sex-hormone related disease or condition” encompasses diseases or conditions involving the reproductive system, and/or which are dependent upon a sex hormone, i.e. a male hormone or female hormone. In one embodiment, these include diseases or conditions occurring due to an excess of such hormones in mammals or non-mammalian vertebrates (e.g. human, monkey, bovine, horse, dog, cat, sheep, rabbit, rat, mouse, fish etc.). In another embodiment, the diseases/conditions involve cells that carry GnRH receptors.

Thus, in one embodiment, [D-Lys⁶(Emo)]GnRH, or [D-Lys⁶(Emo)]GnRH-Antg is useful for the prevention or treatment of sex hormone-dependent malignant diseases, such as cancer. Non-limiting examples include prostate cancer, breast cancer, ovarian cancer, cervical cancer, tumor of the pituitary, testicular cancer, and uterine cancer.

In another embodiment, [D-Lys⁶(Emo)]GnRH or [D-Lys⁶(Emo)]GnRH-Antg is useful for the prevention or treatment of sex hormone-dependent non-malignant (benign) diseases or conditions. Non-limiting examples include benign prostatic hyperplasia, precocious puberty, aberrant sexual behavior (treatment by chemical castration), late luteal phase dysphoric disorder (premenstrual syndrome), fibroids, endometriosis, myoma, hirsutism, cyclic auditory dysfunction, porphyria, or polycystic ovarian syndrome.

As used herein, the term “treating” means remedial treatment, and encompasses the terms “reducing”, “suppressing” “ameliorating” and “inhibiting”, which have their commonly understood meaning of lessening or decreasing. The term “preventing” means inhibiting the disease or condition, so that the disease or condition does not develop or progress.

Furthermore, in another embodiment, the present invention provides a method of promoting the release of LH and FSH in a subject, the method comprising the step of administering to the subject a gonadotropin releasing hormone (GnRH) agonist peptide having the formula (D-Lys⁶(Emo)]GnRH, or a pharmaceutically acceptable salt or hydrate thereof, in an amount effective to promote the release of LH and FSH in the subject.

Furthermore, in another embodiment, the present invention provides a method of preventing suppressing the release of LH and FSH in a subject, the method comprising the step of administering to the subject a long-acting gonadotropin releasing hormone (GnRH) agonist or antagonist peptide having the formula [D-Lys⁶(Emo)]GnRH or [D-Lys⁶(Emo)]GnRH-Antg or a pharmaceutically acceptable salt or hydrate thereof, in an amount effective to prevent the release of LH and FSH in the subject.

In one embodiment, the present invention comprises administering a pharmaceutically acceptable salt of the [D-Lys⁶(Emo)]GnRH or [D-Lys⁶(Emo)]GnRH-Antg peptide. The term “pharmaceutically acceptable salt” includes acid addition salts which are formed with inorganic acids such as, for example, hydrochloric or phosphoric acids, or such organic acids as acetic, oxalic, tartaric, mandelic, and the like. The term also includes base addition salts which are formed from inorganic bases such as, for example, sodium, potassium, ammonium, and calcium, and from organic bases such as isopropylamine, trimethylamine, histidine, and the like.

In another embodiment, the present invention comprises administering a hydrate of the [D-Lys⁶(Emo)]GnRH or [D-Lys⁶(Emo)]GnRH-Antg peptide. The term “hydrate” includes but is not limited to hemihydrate, monohydrate, dihydrate, trihydrate and the like.

In another embodiment, the methods of the present invention comprise administering a pharmaceutical preparation comprising the GnRH agonist or antagonist peptide, and a pharmaceutically acceptable carrier. As used herein, “pharmaceutical preparation” or “pharmaceutical compositions”, used herein interchangeably, means a “therapeutically effective amount” of the [D-Lys⁶(Emo)]GnRH or [D-Lys⁶(Emo)]GnRHAntg. A “therapeutically effective amount” as used herein refers to that amount which provides a therapeutic effect for a given condition and administration regimen.

In one embodiment, pharmaceutical preparation is orally administered in solid or liquid dosage form. In another embodiment, the pharmaceutical preparation is intravenously, intraarterially, subcutaneously, intradermally, intraperitoneally, intramuscularly or intralesionally injected in liquid form. In another embodiment, the pharmaceutical preparation is administered as an intravaginal ring. In another embodiment, the pharmaceutical preparation is administered intranasally. In another embodiment, the pharmaceutical preparation is formulated as a topical formulation for topical application. In another embodiment, the pharmaceutical preparation is a pellet, a tablet, a capsule, a solution, a suspension, an emulsion, a gel, a cream, a suppository, an intra-vaginal ring, or a parenteral formulation. In a particular embodiment the pharmaceutical preparation is formulated as a depot for providing sustained release of the active ingredient. In another particular embodiment the pharmaceutical preparation is formulated for intranasal administration or inhalation.

In one embodiment, the pharmaceutical preparations are administered orally, and are thus formulated in a form suitable for oral administration, i.e. as a solid or a liquid preparation. Suitable solid oral formulations include tablets, capsules, pellets and the like. Suitable liquid oral formulations include solutions, suspensions, emulsions, and the like.

Further, in another embodiment, the pharmaceutical preparations are administered by intravenous, intraarterial, intraperitoneal, subcutaneous, intradermal, intramuscular or injection of a liquid preparation. Suitable liquid formulations include solutions, suspensions, emulsions, and the like. Alternative embodiments include depots providing sustained release or prolonged duration of activity of the active ingredient in the subject, as are well known in the art.

Further, in another embodiment, the pharmaceutical compositions are administered topically to body surfaces, and are thus formulated in a form suitable for topical administration. Suitable topical formulations include gels, ointments, creams, lotions, and the like. Further, in another embodiment, the pharmaceutical compositions are administered as a suppository, for example a rectal suppository or a vaginal or urethral suppository. Further, in another embodiment, the pharmaceutical compositions can be applied on conventional intravaginal rings or other intravaginal devices. Further, in another embodiment, the pharmaceutical compositions can be are administered intranasally or by inhalation.

As used herein a “pharmaceutically acceptable carrier” may be a solid carrier for solid formulations, a liquid carrier for liquid formulations, or mixtures thereof. Solid carriers include, but are not limited to, a gum, a starch (e.g. corn starch, pregelatinized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof. For liquid formulations, pharmaceutically acceptable carriers may be aqueous or non-aqueous solutions, suspensions, or emulsions. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media.

Parenteral vehicles (for subcutaneous, intravenous, intraarterial, intraperitoneal or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions.

In addition, the compositions may further comprise binders (e.g. cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl, acetate, phosphate) of various pH and ionic strength, additives such as gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, the pharmaceutical compositions provided herein are controlled release compositions, i.e. compositions in which the [D-Lys⁶(Emo)]GnRH or [D-Lys⁶(Emo)]GnRH-Antg peptide is released over a period of time after administration. In another embodiment, the composition is an immediate release composition, i.e. a composition in which all of the [D-Lys⁶(Emo)]GnRH or [D-Lys⁶(Emo)]GnRH-Antg. peptide is released immediately after administration.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, the [D-Lys⁶(Emo)]GnRH or [D-Lys⁶(Emo)]GnRH-Antg peptide is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the [D-Lys⁶(Emo)]GnRH or [D-Lys⁶(Emo)]GnRH-Antg peptide is converted into a solution, suspension, or emulsion, if desired with the substances customary and suitable for this purpose, for example, solubilizers or other.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLE 1

### Synthesis of [D-Lys₆]GnRH Conjugates

The synthesis of [D-Lys⁶]GnRH conjugates that are modified at the ε-amino group of [D-Lys⁶]GnRH was carried out by two different routes. The first route involved the reaction of the free ε-amino group of solid-phase synthesized [D-Lys⁶]GnRH, in a homogeneous solution, with the carboxylic functional group of the respective quinone moieties. This method employed benzotriazole-1-yl-oxy-tris-pyrrolidino-phosphonium hexafluorophosphate (PyBOP) as a coupling reagent and 4-methylmorpholine (NMM) as a base. Such a method is a ‘one-pot synthesis’, which results in better yield and purity than other methodologies that use active esters, such as N-hydroxysuccinimide, as coupling reagents¹⁹.

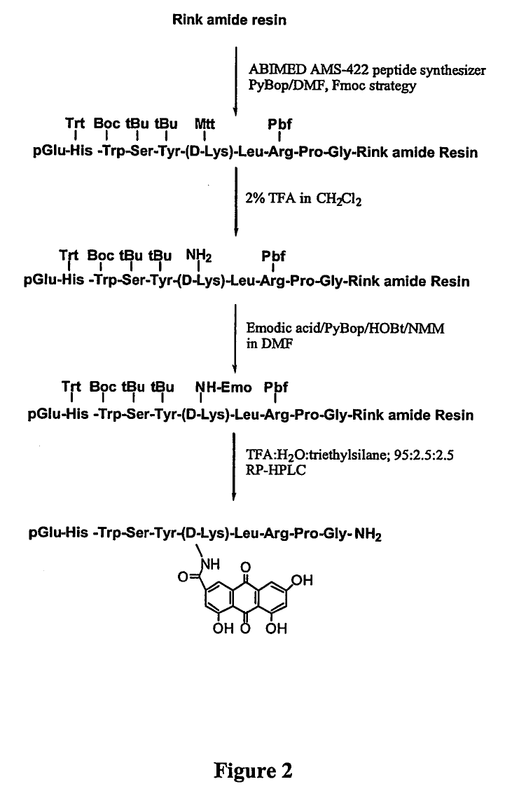

In the second route, [D-Lys⁶]GnRH derivatives were prepared by employing an automatic multiple peptide synthesizer, using Rink amide resin as the polymeric support, and standard Fmoc-protected amino acids and corresponding reagents (FIG. 2). The routinely used Fmoc-D-Lys(Boc)-OH was replaced by Fmoc-D-Lys(Mtt)-OH and the protected peptide was not cleaved from the resin. The Mtt group was removed selectively from the N^{ε}-amino group of D-Lys⁶ by mild acidolysis (2% TFA in CH₂Cl₂) without effecting other protecting groups²⁰. Consequently, direct incorporation of emodic acid (Emo), 2-hydroxymethyl-anthraquinone hemiglutarate (AntrQ), and N-(2-chloro-1,4-naphthoquinonyl)-β-alanine (NQ) (FIG. 1) to the free N^{ε}-amino function of D-Lys⁶ occurs by employing the standard coupling reagent and procedure. This reaction leads to the synthesis of [D-Lys⁶(1,3,8-trihydroxy-6-carboxy-anthraquinone)]GnRH ([D-Lys⁶(Emo)]GnRH), [D-Lys⁶(2-hydroxymethyl-anthra-quinone hemiglutarate)] GnRH ([D-Lys⁶(AntrQ)]GnRH) and [D-Lys⁶(N-(2-chloro-1,4-naphthoquinonyl)-β-alanyl)]GnRH ([D-Lys⁶(NQ)]GnRH).

The naphthoquinone and anthraquinone derivatives were synthesized in order to evaluate the generality of the synthetic manipulations and as a reference for the biological activity of other conjugates of [D-Lys⁶]GnRH. The corresponding molecular mass and the relative hydrophobicity of each of the conjugates are summarized in Table 1.

TABLE 1
GnRH analogs: molecular mass and relative hydrophobicity
and binding affinity (IC₅₀) to rat pituitary receptors.

| | Hydrophobicity | MH⁺ |
|---|---|---|
| Peptide analog | (% B)^{a} | obsd (calcd)^{b} |
| [D-Lys⁶]GnRH | 37 | 1254.6 |
| | | (1254.4) |
| [D-Lys⁶(NQ)]GnRH | 59 | 1516 |
| | | (1516.07) |
| [D- | 84 | 1589.7 |
| Lys⁶(AntrQ)]GnRH | | (1588.7) |
| [D- | 66 | 1537.6 |
| Lys⁶(Emo)]GnRH | | (1536.7) |

^{a}The percent of buffer B in which the analog was eluted from RP-18 column.
^{b}Observed (obsd) and calculated (calcd) m/z values of MH⁺ monoisotopes. In most cases, an additional peak, corresponding to MNa⁺, was observed. Purities of the synthetic peptides were usually >98%, according to two different analytical HPLC solvent systems as detailed in the Experimental Section. For more details and abbreviations see FIGS. 1 and 2.

### EXAMPLE 2

### In Vitro Binding of [D-Lys₆]GnRH to GnRH Receptors and LH-Releasing Activity

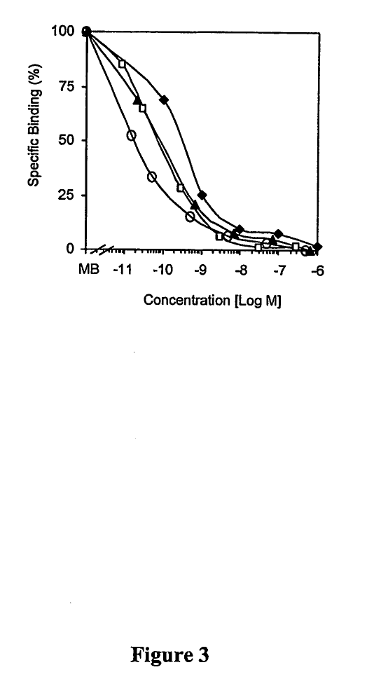

The ability of the [D-Lys⁶]GnRH conjugates to bind to rat pituitary GnRH receptors was evaluated in vitro by displacement assays, using ¹²⁵I[D-Lys⁶]GnRH as the radioligand. FIG. 3 shows that incorporation of the quinone moiety in position 6 of the GnRH analog does not change the high binding affinity of the parent peptide to the GnRH receptors. Moreover, the binding affinity of [D-Lys⁶(NQ)]GnRH and of [D-Lys⁶(AntrQ)]GnRH to GnRH receptors increased by 2 and 5 folds, respectively, whereas the affinity of [D-Lys⁶(Emo)]GnRH to the GnRH receptors was reduced by about 3 fold compared to the parent peptide. The binding affinities (IC₅₀) of the GnRH conjugates are summarized in Table 2.

TABLE 2
GnRH analogs: binding affinity (IC₅₀) to rat pituitary receptors.

| | GnRH |
|---|---|
| | receptor |
| | binding |
| Peptide analog | IC₅₀ (×10⁻¹⁰M) |
| [D-Lys⁶]GnRH | 0.8 ± 0.01 |
| [D-Lys⁶(NQ)]GnRH | 0.9 ± 0.01 |
| [D-Lys⁶(AntrQ)]GnRH | 0.2 ± 0.02 |
| [D-Lys⁶(Emo)]GnRH | 2.5 ± 0.1 |

IC₅₀ = concentration of unlabeled ligand that displaces 50% of bound tracer. Each point is the mean ± SEM of triplicates of one experiment out of three. For more details and abbreviations see FIGS. 1 and 3.

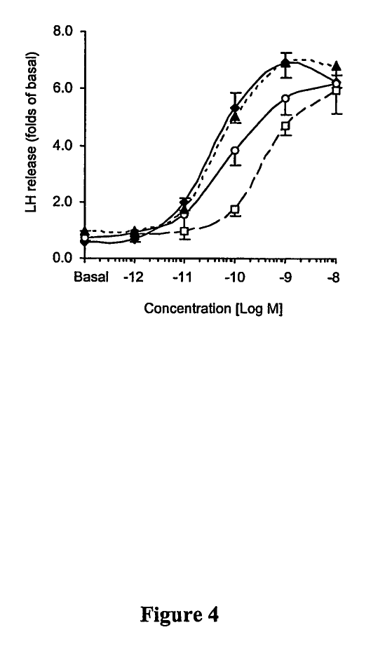

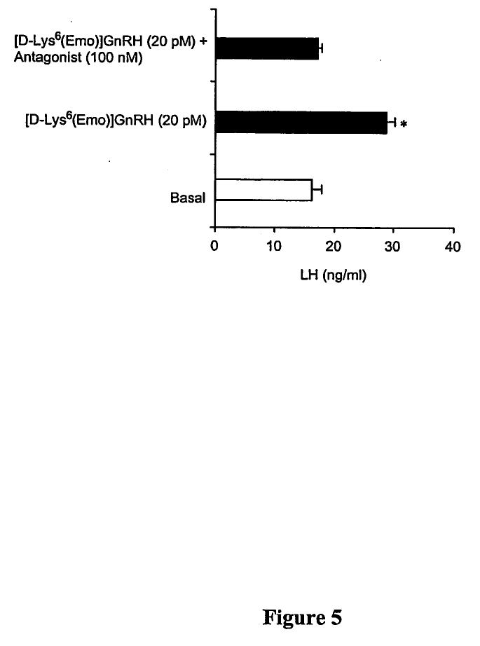

In order to correlate the binding affinity of the newly synthesized GnRH conjugates to their bioactivity, these compounds were compared to [D-Lys⁶]GnRH for the LH-releasing capacity, using primary rat pituitary cell cultures. As shown in FIG. 4, all conjugates exhibited enhanced LH releasing activity compared to the parent peptide, in vitro. Notably, [D-Lys⁶(Emo)]GnRH demonstrated the highest bioactivity, despite its lowest binding affinity to the GnRH receptors (FIG. 3). This bioactivity was completely inhibited by the antagonist [D-pGlu¹, D-Phe², D-Trp^{3,6}]GnRH, which reduced the [D-Lys⁶(Emo)]GnRH-induced LH-secretion to basal levels (FIG. 5).

In conclusion, the in-vitro studies demonstrated a correlation between the binding affinities to GnRH receptors and the in vitro LH releasing potencies of the [D-Lys⁶(NQ)]GnRH and [D-Lys⁶(AntrQ)]GnRH conjugates. [D-Lys⁶(Emo)]GnRH, however, exhibited high LH-releasing potency despite its relatively low binding affinity. The gonadotropin-releasing activity of this conjugate could be completely inhibited by the antagonist, [D-Pyr¹, D-Phe², D-Trp^{3,6}]GnRH, indicating that this activity is receptor mediated.

Furthermore, as demonstrated herein, the in vitro LH-releasing potency of [D-Lys⁶(Emo)]GnRH was superior to that of other conjugates despite its substantial lower affinity to rat pituitary GnRH receptors (FIGS. 3 and 4). This discrepancy might, perhaps, stem from the fact that the nature of its peptide-receptor binding may be different from that of the other derivatives, due to a combination of enhanced hydrophobicity (Table 1) and high capacity to form hydrogen bonds through the hydroxylic groups of emodic acid moiety. Such interactions may reduce the binding affinity but enhance receptor activation.

### EXAMPLE 3

### In Vivo Agonistic Activity of [D-Lys₆]GnRH

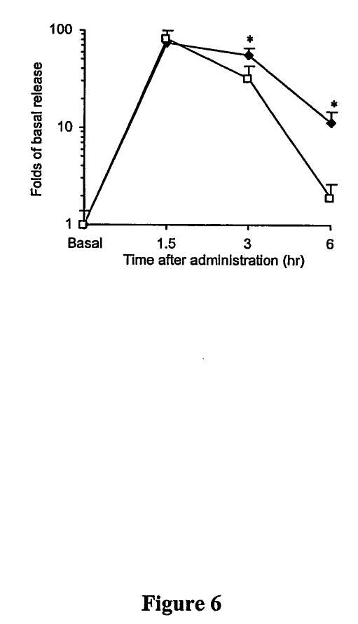

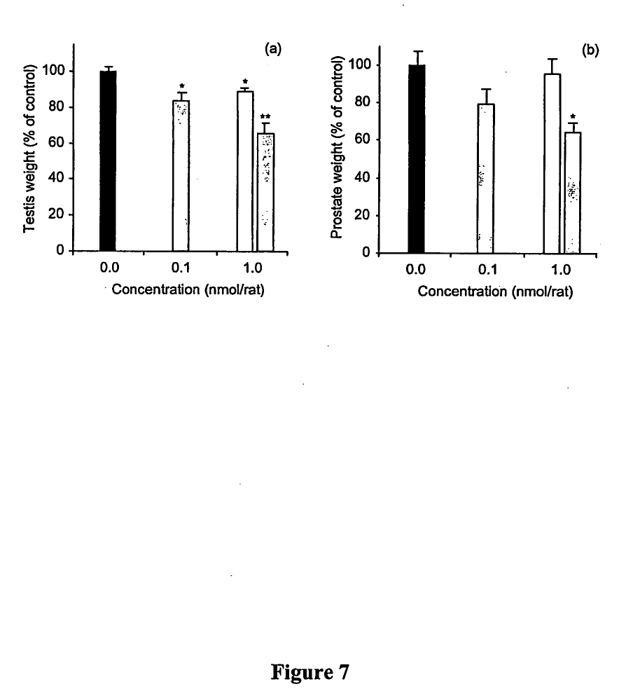

The ability of [D-Lys⁶(Emo)]GnRH to bind GnRH receptors and promote LH release in vitro, prompted the evaluation of its in vivo activity. Following intraperitoneal administration to intact rats, this conjugate proved to be a very potent agonist which induced a similar increase in serum LH levels as that of [D-Lys⁶)GnRH, although the dose was reduced to 10% of that of the parent peptide (0.04 vs. 0.4 nmol) (FIG. 6). Moreover, the duration of the stimulation was also longer; six h after [D-Lys⁶(Emo)]GnRH administration, LH levels were about 6 folds higher than in the group of rats treated with [D-Lys⁶]GnRH. Administration of a higher dose of [D-Lys⁶]GnRH (20 nmol/rat) induced a larger increase in serum LH levels. Furthermore, chronic administration of [D-Lys⁶(Emo)]GnRH (0.1 nmol/rat) to intact adult male rats for 7 days resulted in a greater decrease in the testicular and ventral prostate gland weights as compared to rats that were treated with 10-fold higher dose of the parent peptide (1 nmol/rat) (FIG. 7).

In conclusion, the in-vivo studies demonstrated that [D-Lys⁶(Emo)]GnRH is a powerful long acting agonist, which preserves its activity significantly longer than the parent peptide [D-Lys⁶]GnRH. Moreover, chronic treatment of adult males rats showed that [D-Lys⁶(Emo)]GnRH is much more active in reducing the weights of testes and prostate gland than the parent peptide.

### EXAMPLE 4

### Binding of [D-Lys₆]GnRH to HSA

Binding of peptides to serum proteins, such as human serum albumin (HSA), has been proposed to prolong activity of the peptides^{10,18}. Albumin, the most abundant protein in human serum (with the concentration of about 0.6 mM), possesses a half-life in circulation of 19 days. Its role in the circulatory system is probably to aid in the transport, metabolism, and distribution of exogenous and endogenous ligands²⁶.

As indicated earlier, emodin is known to bind to (HSA). In order to determine whether the prolonged in vivo activity of [D-Lys⁶(Emo)]GnRH might be related to its binding to plasma proteins, its association with HSA was measured.

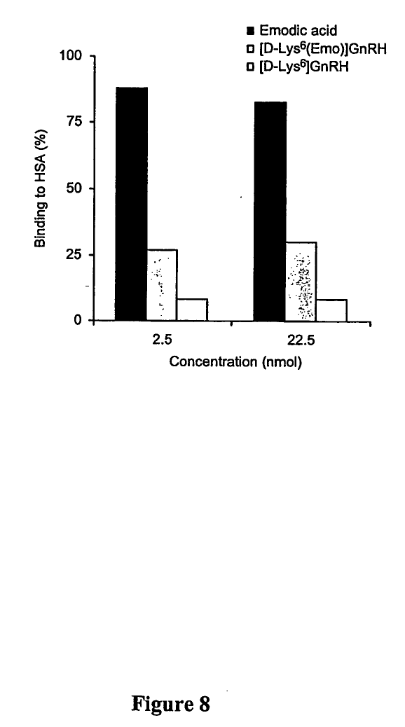

As shown in FIG. 8, emodic acid binds to HSA at the range of the concentrations that were examined. Consequently, the conjugation of emodic acid to [D-Lys⁶]GnRH generates a superagonist, [D-(Emo)]GnRH, which binds significantly to HSA, and thus may explain its prolonged in vivo activity.

Development of potent, long acting GnRH analogs is of particular interest, since in the clinic GnRH analogs are frequently administered in slow-release depot preparations in order to desensitize the pituitary gland. The HSA-binding studies demonstrate that the long-term bioactivity of [D-Lys⁶(Emo)]GnRH may be attributed, at least partly, to the high binding affinity of the emodin moiety to serum proteins, which may protect the peptide from proteolytic degradation.

### EXAMPLE 5

### In Vitro Toxicity Measurement

Emodin is known to be a photoactive compound that generates reactive oxygen species upon illumination^{21,22}. therefore its cytotoxicity as well as that of [D-(Emo)]GnRH to a mouse gonadotroph cell line (αT3-1) that expresses high affinity binding sites for GnRH23, was measured.

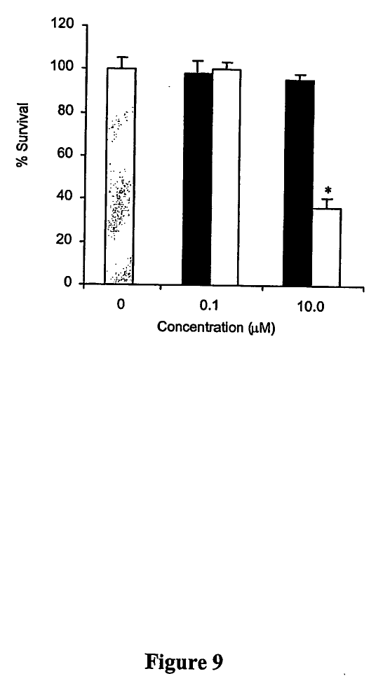

As shown in FIG. 9, although emodic acid demonstrated significant phototoxicity to αT3-1 cells at a concentration of 10 μM, its conjugate, [D-Lys⁶ (Emo)]GnRH, did not exhibit any toxicity. Both compounds did not show any toxicity to these cells while incubated in the dark.

To further ensure that [D-Lys⁶(Emo)]GnRH is not toxic to pituitary cells, emodic acid and [D-Lys⁶(Emo)]GnRH were tested for their ability to induce an apoptotic-like process in αT3-1 cells. These experiments were carried out in dark conditions as well as under illumination.

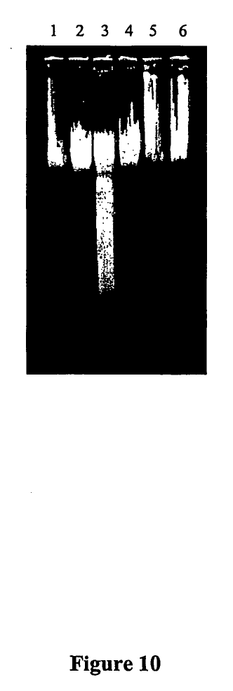

As shown in FIG. 10 emodic acid and its GnRH analog did not cause any DNA fragmentation in the dark (lanes 4 and 6), while upon illumination, only emodic acid (lane 3) but not [D-Lys⁶(Emo)]GnRH (lane 5) induced DNA fragmentation.

In conclusion, the assumption that the high LH releasing activity of [D-Lys⁶(Emo)]GnRH may result from its cytotoxicity and cellular damage, eventually leading to leakage of LH from the gonadotropic cells, was ruled out by evaluation of the toxicity as well as the apoptotic potency of the conjugate. These results clearly revealed that the peptide was devoid of any toxic activity.

Since emodic acid is considered to be a photoactive anthraquinone, these studies were carried out in darkness as well as under illumination. Indeed, the results showed that in the dark neither emodic acid nor [D-Lys⁶(Emo)]GnRH were toxic and could not induce apoptosis to pituitary cell line, while upon illumination emodic acid, but not [D-Lys⁶(Emo)]GnRH, was cytotoxic and induced apoptosis. These results are supported by Applicants' previous studies which revealed that whereas emodic acid generated ROS upon irradiation, [D-Lys⁶(Emo)]GnRH was much less active in this respect²⁴.

### EXAMPLE 6

### Materials and Methods

Abbreviations

Abbreviations of common amino acids are in accordance with the recommendations of IUPAC. Additional abbreviations: AntrQ, 2-(Hydroxymethyl) anthdaquinone; BSA, bovine serum albumin; DMF, N,N′-dimethylformamide; DMSO, dimethylsulfoxide; ED₅₀, concentration of the ligand which indicates 50% of maximal effect. Emo (Emodic acid), 1,3,8-trihydroxy-6-oxy-9,10-anthraquinone; GnRH, gonadotropin-releasing hormone; HPLC, high performance liquid chromatography; IC₅₀, concentration of ligand which displaces 50% of bound tracer; LH, luteinizing hormone; MB, maximal binding; NQ, 2-β-alanyl-1,4-naphthoquinone; PBS, phosphate buffered saline; pGlu, or Pyr, pyroglutamic acid; RIA, radioimmunoassay; SEM, standard error of the mean.

Reagents

All chemicals and reagents were of analytical grade. Rink amide resin, 9-fluorenylmethoxycarbonyl (Fmoc) protected amino acid derivatives, and all the reagents for solid-phase peptide synthesis were purchased from Novabiochem (Läufelfingen, Switzerland). Side-chain protecting groups employed for peptide synthesis were as follows: Arg, 2,2,4,6,7-pentamethyldihydrobenzofuran-5-sulfonyl (Pbf); His, trityl (Trt); D-Lys, 4-methyltrityl (Mtt) or tert-butyloxycarbonyl (Boc); Trp, tert-butyloxycarbonyl (Boc); Ser and Tyr, tert-butyl (tBu). Reversed phase HPLC was performed on a Spectra-Physics SP-8800 liquid chromatography system equipped with an Applied Biosystems 757 variable wavelength absorbance detector. HPLC prepacked columns were: Lichrocart, containing Lichrosorb RP-18 (250×10 mm; 7 μm, Merck, Darmstadt, Germany) for semi-preparative purification and Lichrospher 100 RP-18 (250×4 mm; 5 μm, Merck, Darmstadt, Germany) and wide pore butyl C4 (250×4.6 mm; 5 μm, J. T. Baker Inc., Phillipsburg, N.J.) for analytical purposes. HPLC purification and analysis were achieved by using a linear gradient established between 0.1% trifluoroacetic acid (ITA) in water as buffer A and 0.1% TFA in 75% aqueous acetonitrile as buffer B. Eluent composition was 10-100% B over 40 mlin, using RP-18 column, and a gradient of 0-100% B over 40 min employing a wide pore butyl (C4) column. N-(2noro-1,4-naphtoqumonyl)-β-alanine (NQ), 1,6,8-Trihydroxy-3-carboxylic acid-anthraquinone (Emo) and 2-hydroxymethyl-anthraquinone hemiglutarate (AntrQ) were synthesized as described^{14,24,27} (for chemical structure see FIG. 1).

Animals

Wistar-derived rats were obtained from the Weizmann Institute Animal Resource Center. Experiments were carried out in compliance with the regulations of the Weizmann Institute of Science.

Cells and Culture Conditions

All tissue culture components were purchased from Biological Industries (Beit Haemek, Israel). Mouse pituitary gonadotrope carcinoma cell line (αT3-1) was obtained from Dr. M. Liscovitch, (Dept. of Biological Regulation, Weizmann Inst. of Science) and maintained routinely in Dulbecco's modified Eagle's medium (DMEM) supplemented with 10% fetal calf serum (FCS) and antibiotics (complete medium). Cells were kept at 5% CO₂ atmosphere and 37° C. in a humidified incubator. Primary pituitary cell cultures were prepared from 21 day-old Wistar-derived female rats as described³⁰ and maintained in M-199 containing 10% horse serum and antibiotics and incubated as described above. Irradiations were carried out with a 100 W halogen lamp (Philips, Germany) with an appropriate filter using a band-pass of 320-510 nm and with λₘₐₓ=400 nm. The fluence rate used for the all irradiations was 14.3 mW cm⁻² with a total fluence of 17 J cm⁻².

Synthesis of D-Lys⁶]GnRH] Conjugates

Peptide Synthesis

Automated solid phase peptide synthesis was performed using a multiple peptide synthesizer (AMS-422, Abimed Analysen-Technik GmbH, Langenfeld, Germany) with Rink amide resin (25 μmol scale) as a polymeric support, following the company's protocol for Fmoc strategy^{19,28,29}. For the synthesis of [D-Lys⁶]GnRH conjugates on solid phase support, the Mtt protecting group was used as the side chain protecting group for D-Lys⁶ residue. The completed peptide chain was cleaved from the resin, along with side-chain deprotection, using 3 mL of the mixture TFA:H₂O:triethylsilane; (95:2.5:2.5, v:v), for 2 h at room temperature. The crude products were precipitated with ice-cold tert-butyl methyl ether. Precipitated peptides were washed with cold dry tert-butyl methyl ether, dissolved in water or water/acetonitrile solution, and lyophilized. Peptide purification to homogeneity (usually >96%) was achieved with semi-preparative HPLC and tested by analytical HPLC using the above solvent systems. Samples of each of the peptides were hydrolyzed (6N HCl, 110° C., 22 h, in vacuum) and analyzed with a Dionex automatic amino acid analyzer. The results were also used for quantification of the peptide content in each preparation. The peptides were also analyzed by a Micromass Platform LCZ 4000 (Manchester, UK) using an electron spray ionization technique (ESI). For biological evaluations, pure peptides were dissolved in dimethylsulfoxide (DMSO) or in double distilled water to obtain 1 mM concentration as a stock solution. The DMSO contents in the preparations used for bioassays were always 1% or lower. Identical concentrations of DMSO were tested and found to have no significant effects on receptor binding assays or hormone secretion.

[D-Lys⁶]GnRH Conjugates

Solution Synthesis

[D-Lys⁶]GnRH was automatically synthesized on a multiple peptide synthesizer and lyophilized (purity of crude product was >90%) as described above. To the DMF solution (1 mL) of dry crude peptide (31 mg, 25 μmol) and corresponding quinone (27.5 μmol) in the presence of 4-methylmorpholine (NMM) (8.2 μL, 75 μmol), a DMF solution (0.5 mL) of PyBOP (13 mg, 27.5 μmol) was added. The mixture was stirred for 2 h at room temperature. The progress of the reaction was followed by the disappearance of [D-Lys⁶]GnRH as revealed by analytical HPLC. Upon completion of the reaction the crude peptide was precipitated with ice cold tert-butyl methyl ether (10 mL) and dried. Purification to homogeneity was achieved by semi-preparative HPLC to yield 22.7 mg (15 μmol; 60%) of [D-Lys⁶(N-(2-chloro-1,4-naphtoquinonyl)-β-alanine)]GnRH. Mass spectrometry: found m/z [M+H]⁺ 1516, calcd for C₇₂ H₉₂ ClN₁₉ O₁₆ [M+H⁺ 1516.07. Amino acid analysis after hydrolysis with 6 M HCl at 110° C. for 22 h: Glu 1.00, His 1.00, Ser 0.87, Tyr 0.98, Lys 1, Leu 0.98, Arg 1.05, Pro 1.01, Gly 0.98. Trp was destroyed under the acidic conditions of hydrolysis. In this manner, [D-Lys⁶(AntrQ)]GnRH and [D-Lys⁶(Emo)]GnRH were also synthesized in solution (yield: 65% and 57%, respectively) and characterized. Mass spectrometry: [D-Lys⁶(AntrQ)]GnRH found m/z [M+H]⁺ 1589.7, calcd for C₇₉H₉₈N₁₈O₁₈ [M+H]⁺ 1588.7. [D-Lys⁶(Emo)]GnRH found m/z [M+H]⁺ 1537.6, calcd for C₇₄H₉₀N₁₈O₁₉ [M+H]⁺ 1536.7. Amino acid analysis after hydrolysis with 6 M HCl at 110° C. for 22 h: [D-Lys⁶(AntrQ)]GnRH, Glu 0.99, His 1.01, Ser 0.87, Tyr 0.98, Lys 1.02, Leu 1.02, Arg 1.05, Pro 0.61, Gly 1.06. [D-Lys⁶(Emo)]GnRH, Glu 1.01, His 0.95, Ser 0.88, Tyr 0.97, Lys 1.02, Leu 1.04, Arg 1.04, Pro 0.42, Gly 1.1.

(b) Solid Phase Synthesis

pGlu-His(Trt)-Trp(Boc)-Ser(tBu)-Tyr(tBu)-D-Lys(Mtt)-Leu-Arg(Pbf)-Pro-Gly-[Rink amide resin] (25 μmol), synthesized by a multiple peptide synthesizer, was treated with 2% TFA in CH₂Cl₂ (2 mL×5, 5 min) to cleave the Mtt group from D-Lys²⁰. The removal of the Mtt group was revealed by the ninhydrin test. The resin was then neutralized by 10% N,N′-diisopropylethylamine in CH₂Cl₂ (2 mL×3, 2 min) and washed with CH₂Cl₂ (2 mL×3). Emodic acid (30 mg, 100 μmol) in DMF (0.1 mL) was then coupled to the free N^{ε}-amino group of D-Lys⁶ following the above mentioned company's protocol²⁹. Cleavage of the resulting derivatives, precipitation and purification were carried out as described above. Yield: 31 mg (20 μmol, 80%, based on the initial amino group-resin loading). Mass spectrometry: found m/z [M+H)⁺ 1537.4, calcd for C₇₂H₉₂ClN₁₉O₁₆[M+H]⁺ 1536.7. Similar to this procedure, [D-Lys⁶(AntrQ)]GnRH and [D-Lys⁶(NQ)]GnRH were synthesized on polymer support to yield 85% and 83%, respectively. Mass spectroscopy and amino acid analysis yielded the expected results, similar to those obtained from the synthesis in solution.

In Vitro GnRH Receptor Binding Assay and LH Releasing Potency of [D-Lys⁶]GnRH Derivatives

Displacement binding assays were carried out using rat pituitary membrane preparations and ¹²⁵I[D-Lys⁶]GnRH as radioligand as described³⁰. Briefly, membranes were incubated for 90 min at 4° C. with ¹²⁵I[D-Lys⁶]GnRH and with the unlabeled peptides. Non-specific binding was defined as the residual binding in the presence of excess [D-Lys⁶]GnRH (1 μM). Specific binding was calculated by subtracting the non-specific binding from the maximal binding, determined in the absence of any competing peptide. Results are the mean of two experiments carried out in triplicates. SEM values are omitted for clarity (FIG. 3).

For evaluating the LH releasing potencies of the [D-Lys⁶]GnRH derivatives, rat pituitary cells were incubated in M-199 (without serum and antibiotics) containing the desired concentrations of the tested peptides and incubated in the dark at 37° C. for 4 h as described³¹. The media were then collected and LH concentration were analyzed by double-antibody radioimmunoassay (RIA)³² using kits kindly supplied by the National Institute of Arthritis, Metabolism and Digestive Diseases (NIMDD). Results (FIG. 4) are expressed in terms of LH-RP-3 rat reference preparation, and are the mean±SEM of LH concentration of two experiments (4 wells/experimental group, each). The basal release after 4 h was 9.41±2 ng/mL.

To determine the effect of a GnRH antagonist ([D-pyr¹, D-Phe², D-Trp^{3,6}]GnRH) on the induction of LH secretion from primary cultures of rat pituitary cells stimulated by [D-Lys⁶(Emo)]GnRH, cells were incubated for 4 h at 37° C. with [D-Lys⁶(Emo)]GnRH (20 pM) in the absence or presence of the GnRH antagonist (100 nM). The media were collected and LH concentration was determined by RIA (FIG. 5). Results are the mean±SEM of LH concentrations obtained from two experiments (4 wells/experimental group, each). The LH concentration in each well was determined using three different aliquots of the medium. *LH release was significantly higher (p<0.01) than that of the other groups.

In Vivo LH Release

Female rats (˜250 gr) were intraperitoneally injected with the desired concentration of [D-Lys⁶(Emo)]GnRH (0.04 nmol/rat) or [D-Lys⁶]GnRH (0.4 nmol/rat) in 0.5 mL PBS. Blood samples were withdrawn by cardiac puncture under light ether anesthesia at the indicated time intervals, and serum LH levels were determined by RIA as described above (FIG. 6). Results are the mean±SEM of LH concentrations in the serum of five animals/experimental group. The LH concentration was determined using three different dilutions of each serum sample. Similar results were obtained in two other experiments. LH concentration in unstimulated rats was 0.53 ng/mL serum. *LH release was significantly higher (p<0.01) than in the group treated with [D-Lys⁶]GnRH.

Long-Term Treatment of Animals with GnRH Analogs

Intact adult males rats (260-280 g) were injected daily intraperitoneally with the GnRH analogs [D-Lys⁶(Emo)]GnRH (0.1 or 1 nmol/rat) or with [D-Lys⁶]GnRH (1 nmol/rat) in PBS for 7 days. Control rats were injected daily with 0.5 mL of PBS. Rats were sacrificed 24 h after the last injection, and the testes and prostate glands were immediately dissected and weighed (FIG. 7). Results are the meant±SEM of the weight of the organs (six animals/experimental group). The weight of the testes and prostate gland of the control group were 3.367±0.07 and 0.267±0.02 g, respectively. * Weights are significantly different (P<0.01) from the control. ** Significantly different (P<0.001) from control group or from rats treated with [D-Lys⁶]GnRH (1 nmol/rat).

Binding to HSA

The binding capacity of [D-Lys⁶(Emo)]GnRH, [D-Lys⁶]GnRH and emodic acid to HSA (Sigma, St. Louis, Mo.) was evaluated by incubating various concentrations of the tested compounds with HSA (22.5 nmol, 1.5 mg in 0.5 mL of PBS) at 37° C. for 3 h. The concentration of HSA in the solution was determined using a molar extinction coefficient of 39,000 M⁻¹ cm⁻¹ at 277.5 nm³³. The unbound compound was then separated from the HSA solution by applying it on to a Centricon® concentrator column (Amicon Inc., Beverly, Mass.) with a band pass membrane of 30,000 kD according to the manufacture's protocol. The HSA bound compound (˜20 μL) was then precipitated by adding it to acetonitrile (1 mL). The supernatant was kept and the precipitate was dissolved in PBS (100 μL) and re-precipitated. The combined supernatants were evaporated, the residue was dissolved in water containing 0.1% TFA (buffer A) and analyzed by HPLC to determine the amount of the tested compounds. Results represent the mean of two experiments (FIG. 8).

In Vitro Toxicity Measurements

αT3-1 cells (50,000 cells/well) were plated in 96-well tissue culture plates in 0.1 mL of complete medium. After 24 h the medium was changed and cells were incubated in the same medium (without serum and phenol red) containing different concentrations of the tested compounds for 4 h at 37° C. The cells were then washed (×3) with PBS and illuminated (λₘₐₓ=400 nm) in PBS. The fluence rate was 14.3 mW cm⁻² with a total fluence of 17 J cm⁻². Following illumination the media were replaced by complete medium and the plates were incubated for additional 24 h at 37° C. Cell survival was determined using the XTT (Biological Industries, Beit-Haemek, Israel) kit following the manufacture's protocol. Values are expressed as % survival. 100% survival refers to the survival of cells in the control group that were incubated without any emodic acid derivatives (FIG. 9). *Survival is significantly lower (p<0.001) than in the control group.

DNA Fragmentation Assay

αT3-1 cells (×10⁶/dish) were incubated in phenol red and serum free complete medium (37° C., 5% CO₂) with the tested compounds for 5 h in the dark. Cells were then washed with PBS (×3) and illuminated or remained in darkness as described earlier. The media were then replaced by complete medium and cells were incubated for additional 24 h. DNA was then isolated, using Wizard® genomic DNA purification kit (Promega, Madison, Wis.) following the company's protocol and analyzed by gel electrophoresis (0.4% cross linked agarose, ethidium bromide staining).

### EXAMPLE 7

### Emodic Acid Derivatives of GNRH Antagonist

### Synthesis of [D-Pyr¹, D-Phe², D-Trp³, D-Lys⁶(Emo)]GnRH (denoted herein as [D-Lys⁶(Emo)]Antg)

For the synthesis of [D-Pyr¹, D-Phe², D-Trp³, D-Lys⁶(Emo)]GnRH, the crude antagonist, [(D-Pyr¹, D-Phe², D-Trp³, D-Lys⁶)GnRH, (D-Lys⁶)Antg] of GnRH were automatically synthesized on a multiple peptide synthesizer. The crude peptides were cleaved from the resin, precipitated, dissolved in H₂O and lyophilized. The conjugation of Emodic acid to the peptides were carried out in a one pot reaction, using benzotriazole-1-yl-oxy-tris-pyrrolidino-phosphonium hexafluorophosphate (PyBOP) as a coupling agent, 4-methylmorpholine (NMM) as a base and DMF as a solvent for 1 h. The crude conjugates were then purified to homogeneity by semi-preparative HPLC and characterized by UV, MS and amino acid analysis.

Synthesis of [D-Lys⁶(Emo)]Antg was also carried out employing solid phase peptide synthesis, similar to that of [D-Lys⁶(Emo)]GnRH.

Binding of the ([D-Lys⁶(Emo)]Antg) to the pituitary GnRH receptor.

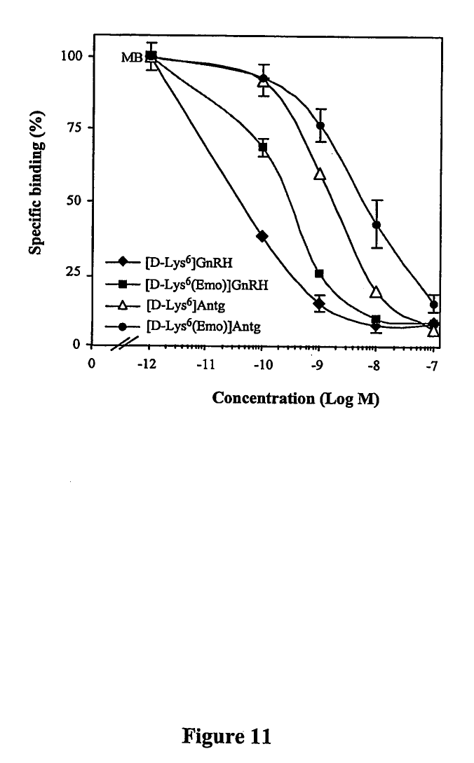

The binding affinities of different GnRH conjugates to the pituitary GnRH receptors were compared by competitive binding experiments as described for [D-Lys⁶(Emo)]GnRH, using ¹²⁵I-[D-Lys⁶]GnRH as a tracer. [D-Lys⁶(Emo)]Antg showed lower binding affinity to GnRH receptors as compared to the parent peptide or to the [D-Lys⁶]GnRH conjugates (FIG. 11). This seems to be mainly due to the similar, low binding affinity of the parent antagonist to GnRH receptors (FIG. 11). It should be noted that the antagonist, [D-Pyr¹, D-Phe², D-Trp³, D-Lys⁶]GnRH, has a lower binding affinity to GnRH receptor as compared to [D-Lys⁶]GnRH and the insertion of emodic acid to either peptides causes a similar loss of binding affinity. FIG. 11 compares the binding affinities of agonistic and antagonistic derivatives of emodin and Table 3 summarizes the estimated IC₅₀ (concentration of the competitor which displace 50% of the bound tracer) of different GnRH analogs to the GnRH receptor.

These results indicate that conjugated analogs of GnRH, agonists as well as antagonists, preserved the binding capacity of their carriers

TABLE 3
The IC₅₀ of GnRH analogs and their emodic acid conjugates.

| Peptide analog | IC₅₀ (M) |
|---|---|
| [D-Lys⁶]-GnRH | 0.7-1.4 × 10⁻⁹ |
| [D-Lys⁶(Emo)]GnRH | 2.6 × 10⁻⁹ |
| [D-Lys⁶]Antg* | 4.5 × 10⁻⁸ |
| [D-Lys⁶(Emo)]Antg | 1.5 × 10⁻⁷ |

*[D-Lys⁶]-Antg correspond to the antagonist, [D-Pyr¹, D-Phe², D-Trp³, D-Lys⁶]GnRH.

The Biological Potencies of the GnRH Conjugates.

To correlate the binding affinity with the bioactivity, we compared the effect of each of the GnRH conjugates with its parent peptide on LH secretion, using primary rat pituitary cell cultures.

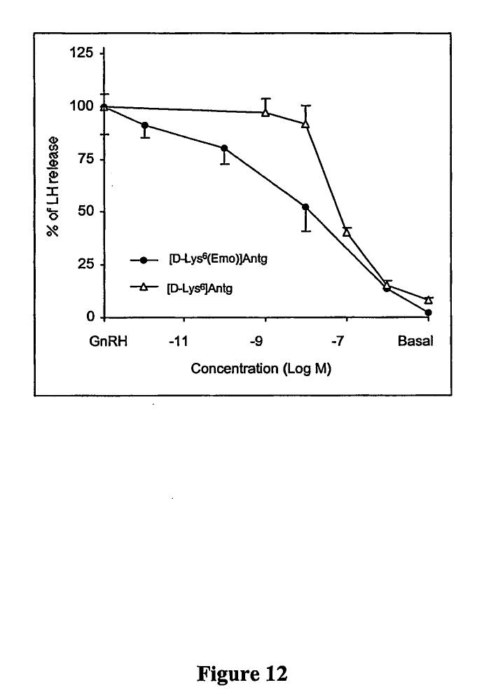

FIG. 12 demonstrates the inhibition of LH secretion from rat pituitary cultures by the two antagonists. Incorporation of emodic acid to the antagonist decreases its binding affinity (FIG. 11) however its bioactivity is superior as compared to the parent antagonist (FIG. 12).

### EXAMPLE 8

### Long Acting GNRH Analogs in Animal Husbandry

In animal husbandry, the management of fertility can be both difficult and extremely important for the success of agricultural or other businesses. Stimulation of ovulation at appropriate times, as well as the induction of cyclicity in some species of domesticated animals that can become seasonally nonovulatory would result in increased management efficiency for these species.

For example, in the husbandry of horses, the development of an accurate, economical method for the precise control of ovulation in the mare would greatly benefit reproductive management of mares and stallions. The mares' extended estrus period, with ovulation at any time from 1 to 10 days after the beginning of estrus, has made reproductive management of mares time-consuming, expensive and most importantly, inefficient. In the mare, GnRH or its analogs are beginning to be used as alternative non-antigenic substitutes to replace hCG to hasten ovulation in preovulatory mares. This is because repeated use of hCG has been associated with decreased response [Sullivan, J., J. Am. Vet. Med. Assoc. 63:895(1973)] and anti-hCG antibody formation [Roser, J., J. Reprod. Fert. Suppl. 173-179(1974)]. Current data suggest that ovulation induction with potent GnRH analogs requires multiple injections of very low doses [Harrison, L., et al., J. Eq. Vet Sci. 11:163-166(1991)] or a very high dose given as a slow releasing implant [Jochle, W. et al., J. Eq. Vet. Sci. 44:632(1994)].

Similar concerns and needs occur in other areas involving the raising and breeding of mammals, in particular livestock mammals, including, for example, the swine and cattle husbandry industries, where control of the estrus of sows and gilts and of cows and heifers, respectively, also would benefit their reproductive management.

In particular, in commercial swine production, maximizing reproductive efficiency offers producers substantial opportunities to reduce production costs and enhance profitability. Currently, a precise method of determining the time of ovulation in spontaneously cycling gilts is not available. In gilts and sows, GnRH affects the synchronization of ovulation. However, at present the variation in time of the onset of ovulation is large enough that two inseminations are required for maximal fertilization. Therefore, use of a long acting GnRH that could stimulate an LH surge capable of reducing the time span of ovulations so that a single timed insemination could be used would greatly benefit reproductive management of gilts, sows, and consequently, boars.

In cattle production, maximizing reproductive efficiency offers producers substantial opportunities to reduce production costs and enhance profitability. This is particularly true in the heifer, due to difficulties in synchronizing estrus compared with cows, a factor that reduces overall herd performance. Therefore, methods to synchronize estrus which increase the level of response and reduce variability would allow management to bring replacement heifers into the herd at lower cost and significantly impact the efficiency of beef and dairy production.

Progress toward reducing reliance on estrus detection for managing reproduction in dairy heifers and cows is being realized by combining timed artificial insemination (AI) with a protocol for synchronization of ovulation that can be initiated at a random stage of the estrous cycle. [Pursley, J. R., et al., Theriogenology 44:915-923 (1995); Pursley, J. R., et al., J. Dairy Sci. 80:295-300 (1997)]. This protocol, commonly called OVSYNCH, synchronizes follicular development, luteal regression and time of ovulation, thereby allowing for timed insemination 12 to 24 hours after the completion of the GnRH/PGF/GnRH treatment protocol. However, hormone cost per treated cow can be significant. [Frick, P. M., et al., Theriogenology 50:1275-1284 (1998)]. Since retail cost of GnRH constitutes the majority of the cost in using OVSYNCH, availability of a cost effective long acting GnRH that could be used in the OVSYNCH protocol would greatly benefit reproductive management of heifers and cows.

Lastly, aquaculture is the fastest growing section of the agricultural industry in many countries. However, many fish do not spawn when raised in captivity & techniques to induce spawning in fish are inefficient due to the lack of methods for controlled administration., availability of an effective long acting GnRH that could he used to induce spawning in fish would greatly benefit reproductive management in aquaculture.

It will be appreciated by a person skilled in the art that the present invention is not limited by what has been particularly shown and described hereinabove. Rather, the scope of the invention is defined by the claims which follow:

### REFERENCE

(1) Schally, A. V.; Arimura, A.; Kastin, A. J.; Matsuo, H.; Baba, Y.; Redding, T. W.: Nair, R. M. G.; Debeljuk, L.; White, W. F. Gonadotropin-releasing hormone: One polypeptide regulates secretion of luteinizing hormone and follicle-stimulating hormones. Science 1971, 173, 1036-1038.

(2) Burgus, R.; Butcher, M.; Amoss, M.; Ling, N.; Monahan, M.; Rivier, J.; Fellows, R; Blackwell, R.; Vale, W.; Guillemin, R. Primary structure of the ovine hypothalamic luteinizing hormone-releasing factor (LRF) (LH-hypothalamus-LRF-gas chromatography-mass spectrometry-decapeptide-Edman degradation). Proc. Natl. Acad. Sci. U.S.A. 1972, 69,278-282.

(3) Belchetz, P. E.; Plant, T. M.; Nakai, Y.; Keogh, E. J.; Knobil, E. Hypophyseal responses to continuous and intermittent delivery of hypothalamic gonadotropin-releasing hormone. Science 1978, 202, 631-632.

(4) Hazum, E.; Conn, P. M. Molecular mechanism of gonadotropin releasing hormone (Gn33) action. I. The GnRH receptor. Endocr. Rev. 1988, 9, 379-386.

(5) Schally, A. V. Luteinizing hormone-releasing hormone analogs: their impact on the control of tumorigenesis. Peptides 1999, 20, 1247-1262.

(6) Nillius, S. J.; Bergquist, C.; Gudmundsson J. A.; Wide, L. Superagonists of LHRH for contraception in women. In LHRH and its analogues; Labrie, F.; Belanger, A.; Dupont, A., Eds.; Elsevier Science Publishers B.V.: Amsterdam, 1984; pp. 261-274.

(7) Foekens, J. A.; Klijn, J. G. M. Direct antitumor effects of LH-RH analogs. Rec. Results Cancer Res. 1992, 124, 7-17.

(8) Karten, M. J.; Rivier, J. E. Gonadotropin-releasing hormone analog design. Structure-function studies toward the development of agonists and antagonists: rationale and perspective. Endocr. Rev. 1986, 7, 44-66.

(9) Koch, Y.; Baram, T.; Hazum, E.; Fridkin, M. Resistance to enzymic degradation of LH-RH analogues possessing increased biological activity. Biochem. Biophys. Res. Commun. 1977, 74, 488-491.

(10) Barron, J. L.; Millar, R. P.; Searle, D. Metabolic clearance and plasma half-disappearance time of D-Trp⁶-LRH and exogenous LRH. J. Clin. Endocrinol. Metab. 1982, 54; 1169-1174.

(11) Meidan, R.; Koch, Y. Binding of luteinizing hormone-releasing hormone analogues to dispersed rat pituitary cells. Life Sci. 1981, 28, 1961-1967.

(12) Hazum, E.; Meidan, R.; Liscovitch, M.; Keinan, D.; Lindner, H. R.; Koch, Y. Receptor-mediated internalization of LHRH antagonists by pituitary cells. Mol. Cell. Endocrinol. 1983, 30, 291-301.

(13) Bajusz, S.; Janaky, T.; Csernus, V. J.; Bokser, L.; Fekete, M.; Srkalovic, G.; Redding, T. W.; Schally, A. V. Highly potent analogues of luteinizing hormone-releasing hormone containing D-phenylalanine nitrogen mustard in position 6. Proc. Natl. Acad. Sci. U.S.A. 1989, 86, 6318-6322.

(14) Janaky, T.; Juhasz, A.; Bajusz, S.; Csernus, V.; Srkalovic, G.; Bokser, L.; Milovanovic, S.; Redding, T. W.; Rekasi, Z.; Nagy, A.; Schally, A. V. Analogues of luteinizing hormone-releasing hormone containing cytotoxic groups. Proc. Natl. Acad. Sci. U.S.A. 1992, 89, 972-976.

(14a) Rahimipour S, Ben-Aroya N, Fridkin M, Koch Y. Design, synthesis, and evaluation of a long-acting, potent analogue of gonadotropin-releasing hormone. J Med Chem. Oct. 25, 2001;44(22):3645-52.

(15) Nagy, A.; Schally, A. V.; Armatis, P.; Szepeshazi, KY; Halmos, G.; Kovacs, M.; Zarandi, M.; Groot, K; Miyazaki, M.; Jungwirth, A.; Horvath, J. Cytotoxic analogs of luteinizing hormone-releasing hormone containing doxorubicin or 2-pyrrolinodoxorubicin, a derivative 500-1000 times more potent Proc. Natl. Acad. Sci. U.S.A 1996, 93, 7269-7273.

(16) Lin, T. S.; Teicher, B. A.; Sartorelli, A. C. 2-Methylanthraquinone derivatives as potential bioreductive alkylating agents. J. Med. Chem. 1980, 23, 1237-1242.

(17) Liang, J. W.; Hsiu, S. L.; Wu, P. P.; Chao, P. D. Emodin pharmacokinetics in rabbits. Planta. Med. 1995, 61, 406-408.

(18) Kurtzhals, P.; Havelund, S.; Jonassen, I.; Makussen, J. Effect of fatty acids and selected drugs on the albumin binding of a long-acting, acylated insulin analogue. J. Pharm. Sci. 1997, 86, 1365-1368.

(19) Rahimipour, S.; Weiner, L.; Shrestha-Dawadi, P. B.; Bittner, S.; Koch, Y.; Fridkin, M. Cytotoxic peptides: Naphthoquinonyl derivatives of luteinizing hormone-releasing hormone. Lett. Pept. Sci. 1998, 5, 421-427.

(20) Aletras, A.; Barlos, K; Gatos, D.; Koutsogianni, S.; Mamos, P. Preparation of the very acid-sensitive Fmoc-Lys(Mtt)-OH. Application in the synthesis of side-chain to side-chain cyclic peptides and oligolysine cores suitable for the solid-phase assembly of MAPs and TASPs. Int. J. Pept. Protein Res. 1995, 45, 488-496.

(21) Gollnick, K; Held, S.; Marire, D. O.; Braslavsky, S. E. Hydroxyanthraquinones as sensitizers of singlet oxygen reactions: quantum yields of triplet formation and singlet oxygen generation in acetonitrile. J. Photochem. Photobiol. A: 1992, 69, 155-165.

(22) Hartnan, P. E.; Goldstein, M. A. Superoxide generation by photomediated redox cycling of anthraquinones. Environ. Mol. Mutagen. 1989, 14, 42-47.

(23) Anderson, L.; Milligan, G.; Eidne, K. A. Characterization of the gonadotrophin-releasing hormone receptor in alpha T3-1 pituitary gonadotroph cells. J. Endocrinol. 1993, 136, 51-58.

(24) Rahimipour, S.; Bilkis, I.; Péron, V.; Gescheidt, G.; Barbosa, F.; Mazur, Y.; Koch, Y.; Weiner, L.; Fridkin, M. Generation of free radicals by Emodic acid and its [D-Lys⁶]GnRH-conjugate. Photochem. Photobiol. 2001, 74, 226-236.

(25) Powis, G. Free radical formation by antitumor quinones. Free Radic. Biol. Med. 1989, 6, 63-101.

(26) Carter, D. C.; Ho, J. X. Structure of serum albumin. Adv. Protein. Chem. 1994, 45, 153-203.

(27) Rahimipour, S.; Weiner, L.; Fridkin, M.; Shrestha-Dawadi, P. B.; Bittner, S. Novel naphthoquinonyl derivatives: potential structural components for the synthesis of cytotoxic peptides. Lett. Pept. Sci. 1996, 3, 263-274.

(28) Atherton, E.; Sheppard, R. C. Solid phase peptide synthesis—a practical approach; IRL Press: Oxford, England, 1989; pp 203

(29) Gausepohl, H.; Kral, M.; Boulin, C.; Frank R. W. A multiple reaction system for automated simultaneous peptide synthesis. In Peptides 1990, Proceedings of the twenty-first European Peptide Symposium, Giratt, E., Andreu, D. (Eds.); Escom Science Publishers, 1991, pp 206-207.

(30) Liscovitch, M.; Ben-Aroya, N.; Meidan, R.; Koch, Y. A differential effect of trypsin on pituitary gonadotropin-releasing hormone receptors from intact and ovariectomized rats. Evidence for the existence of two distinct receptor populations. Eur. J. Biochem. 1984, 140, 191-197.

(31) Yahalom, D.; Koch, Y.; Ben-Aroya, N.; Fridkin, M. Synthesis and bioactivity of fatty acid-conjugated GnRH derivatives. Life Sci. 1999, 64, 1543-1552.

(32) Daane, T. A.; Parlow, A. F. Periovulatory patterns of rat serum follicle stimulating hormone and luteinizing hormone during the normal estrous cycle: effects of pentobarbital. Endocrinology 1971, 88, 653-667.

(33) Koh, S. W.; Means, G. E. Characterization of a small apolar anion binding site of human serum albumin. Arch. Biochem. Biophys. 1979, 192, 73-79.

1. A method of controlling fertility in a subject, comprising the step of administering to said subject a gonadotropin releasing hormone (GnRH) analog comprising a GnRH peptide conjugated to emodic acid or an emodic acid derivative, or a pharmaceutically acceptable salt or hydrate thereof, in an amount effective to control fertility in said subject.

2. The method of claim 1, wherein the GnRH analog is a GnRH agonist having the formula [D-Lys⁶(Emo)]GnRH, or a pharmaceutically acceptable salt or hydrate thereof, in an amount effective to control fertility in said subject.

3. The method of claim 1, wherein the GnRH analog is a GnRH antagonist having the formula [D-Pyr¹,D-Phe²,D-Trp³,D-Lys⁶(Emo)]GnRH, or a pharmaceutically acceptable salt or hydrate thereof, in an amount effective to control fertility in said subject.

4. The method of claim 1 wherein fertility is controlled to prevent conception in a female subject.

5. The method of claim 1, wherein said subject is a mammal.

6. The method of claim 1, wherein said subject is a human.

7. The method of claim 1, wherein said subject is a non-human mammal.

8. The method of claim 1, wherein said subject is a non-mammalian vertebrate.

9. The method of claim 1, wherein said administering comprises administering a pharmaceutical preparation comprising said GnRH analog, and a pharmaceutically acceptable carrier.

10. The method of claim 9, wherein said administering comprises orally administering said pharmaceutical preparation in solid or liquid dosage form; intravenously, intraarterially, intraperitoneally, subcutaneously, intradermally or intramuscularly injecting said pharmaceutical preparation in liquid form; intravaginally applying said pharmaceutical preparation; administering said pharmaceutical preparation intranasally or by inhalation; or topically applying said pharmaceutical preparation.

11. The method of claim 9, wherein said pharmaceutical preparation is a pellet, a tablet, a capsule, a solution, a suspension, an emulsion, a gel, a cream, a suppository, a vaginal ring, a depot or a parenteral formulation.

12. A method of contraception in a subject, comprising the step of administering to said subject a gonadotropin releasing hormone (GnRH) analog comprising a GnRH peptide conjugated to emodic acid or an emodic acid derivative, or a pharmaceutically acceptable salt or hydrate thereof.

13. The method of claim 12 wherein the GnRH analog is a GnRH agonist peptide having the formula [D-Lys6(Emo)]GnRH, or a pharmaceutically acceptable salt or hydrate thereof.

14. The method of claim 12 wherein the GnRH analog is a GnRH antagonist peptide having the formula [D-Pyr¹,D-Phe²,D-Trp³,D-Lys⁶(Emo)]GnRH, or a pharmaceutically acceptable salt or hydrate thereof.

15. The method of claim 12, wherein said subject is a mammal.

16. The method of claim 12, wherein said subject is a human.

17. The method of claim 12, wherein said subject is a non-human mammal.

18. The method of claim 12, wherein said subject is a female.

19. The method of claim 12, wherein said administering comprises administering a pharmaceutical preparation comprising said GnRH analog, and a pharmaceutically acceptable carrier.

20. The method of claim 19, wherein said administering comprises orally administering said pharmaceutical preparation in solid or liquid dosage form; intravenously, intraarterially, intraperitoneally, subcutaneously, intradermally or intramuscularly injecting said pharmaceutical preparation in liquid form; intravaginally applying said pharmaceutical preparation; administering said pharmaceutical preparation intranasally or by inhalation; or topically applying said pharmaceutical preparation.

21. The method of claim 19, wherein said pharmaceutical preparation is a pellet, a tablet, a capsule, a solution, a suspension, an emulsion, a gel, a cream, a suppository, a vaginal ring, a depot or a parenteral formulation.

22. A method of treating a sex hormone-related disease or condition in a subject, said method comprising the step of administering to said subject a gonadotropin releasing hormone (GnRH) analog comprising a GnRH peptide conjugated to emodic acid or an emodic acid derivative, or a pharmaceutically acceptable salt or hydrate thereof, in an amount effective to treat said disease or condition in said subject.

23. The method of claim 22 wherein the GnRH analog is a GnRH agonist peptide having the formula [D-Lys6(Emo)]GnRH, or a pharmaceutically acceptable salt or hydrate thereof, in an amount effective to treat said disease or condition in said subject.

24. The method of claim 22 wherein the GnRH analog is a GnRH antagonist peptide leaving Lue formula LD-Pyr¹,D-Phe²,D-Trp³,D-Lys⁶(Emo)]GnRH, or a pharmaceutically acceptable salt or hydrate thereof, in an amount effective to treat said disease or condition in said subject.

25. The method of claim 22, wherein said disease or condition is prostate cancer, breast cancer, ovarian cancer, cervical cancer, a tumor of the pituitary, testicular cancer or uterine cancer.

26. The method of claim 22, wherein said disease or condition is a benign disease or condition.

27. The method of claim 26, wherein said disease or condition is benign prostatic hyperplasia, precocious puberty, aberrant sexual behavior, late luteal phase dysphoric disorder (premenstrual syndrome), fibroids, endometriosis, myoma, hirsutism, cyclic auditory dysfunction, porphyria, or polycystic ovarian syndrome.

28. The method of claim 22, wherein said subject is a mammal.

29. The method of claim 22, wherein said subject is a human.

30. The method of claim 22, wherein said subject is a non-human mammal.

31. The method of claim 22, wherein said subject is a male.

32. The method of claim 22, wherein said subject is a female.

33. The method of claim 22, wherein said administering comprises administering a pharmaceutical preparation comprising said GnRH analog, and a pharmaceutically acceptable carrier.

34. The method of claim 33, wherein said administering comprises orally administering said pharmaceutical preparation in solid or liquid dosage form; intravenously, intraarterially, intraperitoneally, subcutaneously, intradermally or intramuscularly injecting said pharmaceutical preparation in liquid form; intravaginally applying said pharmaceutical preparation; administering said pharmaceutical preparation intranasally or by inhalation; or topically applying said pharmaceutical preparation.

35. The method of claim 33, wherein said pharmaceutical preparation is a pellet, a tablet, a capsule, a solution, a suspension, an emulsion, a gel, a cream, a suppository, a vaginal ring, a depot or a parenteral formulation.

36. A method of treating a sex hormone-related disease or condition in a subject, said method comprising the step of administering to said subject a gonadotropin releasing hormone (GnRH) analog comprising a GnRH peptide conjugated to emodic acid or an emodic acid derivative, or a pharmaceutically acceptable salt or hydrate thereof, in an amount effective to treat said disease or condition in said subject.

37. The method of claim 36 wherein the GnRH analog is a GnRH agonist peptide having the formula [D-Lys6(Emo)]GnRH, or a pharmaceutically acceptable salt or hydrate thereof, in an amount effective to treat said disease or condition in said subject.

38. The method of claim 36 wherein the GnRH analog is a GnRH antagonist peptide having the formula [D-Pyr¹,D-Phe²,D-Trp³,D-Lys6 (Emo)]GnRH, or a pharmaceutically acceptable salt or hydrate thereof, in an amount effective to treat said disease or condition in said subject.

39. The method of claim 36, wherein said disease or condition is prostate cancer, breast cancer, ovarian cancer, cervical cancer, a tumor of the pituitary, testicular cancer or uterine cancer.

40. The method of claim 36, wherein said disease or condition is a benign disease or condition.

41. The method of claim 40, wherein said disease or condition is benign prostatic hyperplasia, precocious puberty, aberrant sexual behavior, late luteal phase dysphoric disorder (premenstrual syndrome), fibroids, endometriosis, myoma, hirsutism, cyclic auditory dysfunction, porphyria, or polycystic ovarian syndrome.

42. The method of claim 36, wherein said subject is a mammal.

43. The method of claim 36, wherein said subject is a human.

44. The method of claim 36, wherein said subject is a non-human mammal.

45. The method of claim 36, wherein said subject is a male.

46. The method of claim 36, wherein said administering comprises administering a pharmaceutical preparation comprising said GnRH analog, and a pharmaceutically acceptable carrier.

47. The method of claim 46, wherein said administering comprises orally administering said pharmaceutical preparation in solid or liquid dosage form; intravenously, intraarterially, intraperitoneally, subcutaneously, intradermally or intramuscularly injecting said pharmaceutical preparation in liquid form; intravaginally applying said pharmaceutical preparation; administering said pharmaceutical preparation intranasally or by inhalation; or topically applying said pharmaceutical preparation.

48. The method of claim 46, wherein said pharmaceutical preparation is a pellet, a tablet, a capsule, a solution, a suspension, an emulsion, a gel, a cream, a suppository, a vaginal ring, a depot or a parenteral formulation.

49. A method of promoting the release of LH or FSH in a subject, comprising the step of administering to said subject a gonadotropin releasing hormone (GnRH) analog comprising a GnRH peptide conjugated to emodic acid or an emodic acid derivative, or a pharmaceutically acceptable salt or hydrate thereof, in an amount effect to promote the release of LH or FSH in said subject.

50. The method of claim 49 wherein the GnRH analog is a GnRH agonist peptide having the formula [D-Lys⁶(Emo)]GnRH, or a pharmaceutically acceptable salt or hydrate thereof, in an amount effective to promote the release of LH or FSH in said subject.

51. The method of claim 49, wherein said subject is a mammal.

52. The method of claim 49, wherein said subject is a human.

53. The method of claim 49, wherein said subject is a non-human mammal.

54. The method of claim 49, wherein said subject is a non-mammalian vertebrate.

55. The method of claim 49, wherein said subject is a male.

56. The method of claim 49, wherein said subject is a female.

57. The method of claim 49, wherein said administering comprises administering a pharmaceutical preparation comprising said GnRH analog, and a pharmaceutically acceptable carrier.

58. The method of claim 57, wherein said administering comprises orally administering said pharmaceutical preparation in solid or liquid dosage form; intravenously, intraarterially, intraperitoneally, subcutaneously, intradermally or intramuscularly injecting said pharmaceutical preparation in liquid form; intravaginally applying said pharmaceutical preparation; administering said pharmaceutical preparation intranasally or by inhalation; or topically applying said pharmaceutical preparation.

59. The method of claim 57, wherein said pharmaceutical preparation is a pellet, a tablet, a capsule, a solution, a suspension, an emulsion, a gel, a cream, a suppository, a vaginal ring, a depot or a parenteral formulation.

60. A method of preventing the release of LH or FSH in a subject, comprising the step of administering to said subject a gonadotropin releasing hormone (GnRH) analog comprising a GnRH peptide conjugated to emodic acid or an emodic acid derivative, or a pharmaceutically acceptable salt or hydrate thereof, in an amount effect to prevent the release of LH or FSH in said subject.

61. The method of claim 60 wherein the GnRH analog is a GnRH agonist peptide having the formula [D-Lys⁶(Emo)]GnRH, or a pharmaceutically acceptable salt or hydrate thereof, in an amount effective to prevent the release of LH or FSH in said subject.

62. The method of claim 60 wherein the GnRH analog is a gonadotropin releasing hormone (GnRH) antagonist peptide having the formula [D-Pry¹, D-Phe²,D-Trp³,D-Lys⁶(Emo)]GnRH, or a pharmaceutically acceptable salt or hydrate thereof, in an amount effective to prevent the release of LH or FSH in said subject.

63. The method of claim 60, wherein said subject is a mammal.

64. The method of claim 60, wherein said subject is a human.

65. The method of claim 60, wherein said subject is a non-human mammal or a non-mammalian vertebrate.

66. The method of claim 60, wherein said subject is a male.

67. The method of claim 60 wherein said subject is a female.

68. The method of claim 60, wherein said administering comprises administering a pharmaceutical preparation comprising said GnRH analog, and a pharmaceutically acceptable carrier.

69. The method of claim 68, wherein said administering comprises orally administering said pharmaceutical preparation in solid or liquid dosage form; intravenously, intraarterially, intraperitoneally, subcutaneously, intradermally or intramuscularly injecting said pharmaceutical preparation in liquid form; intravaginally applying said pharmaceutical preparation; administering said pharmaceutical preparation intranasally or by inhalation; or topically applying said pharmaceutical preparation.

70. The method of claim 68, wherein said pharmaceutical preparation is a pellet, a tablet, a capsule, a solution, a suspension, an emulsion, a gel, a cream, a suppository, a vaginal ring, a depot or a parenteral formulation.

71. A long acting gonadotropin releasing hormone (GnRH) analog comprising a GnRH peptide conjugated to emodic acid or an emodic acid derivative, or a pharmaceutically acceptable salt or hydrate thereof, other than D-Lys⁶(Emo)GnRH.

72. A GnRH analog according to claim 71 wherein the analog is an agonist having the formula: Pyr-His-Trp-Y-Tyr-X-Leu-Arg-Pro-Z
wherein X is selected from Ser(Emo), D-Ser(Emo), Lys(Emo), D-Lys(Emo), D-Dab(Emo), D-Orn(Emo), D-hSer(Emo); Y is selected from D-Lys(Emo), D-Dab(Emo), D-Orn(Emo), D-Ser(Emo), D-hSer(Emo); Z is selected from Gly, Ethylamine, D-Ala; Pyr denotes pyroglutamic acid, Dab denotes diaminobutyric acid; Emo denotes emodic acid or an emodic acide derivative; and pharmaceutically acceptable salts, amides, esters and hydrates thereof.

73. A gonadotropin releasing hormone (GnRH) antagonist peptide having the formula:
i) [D-Pyr¹, D-Phe²,D-Trp³,D-Lys⁶(Emo)]GnRH, or a pharmaceutically acceptable salt or hydrate thereof;
ii) Ac-D-Nal-4-chloro-D-Phe-β(3-Pyridyl)-D-Ala-X—Y-Z-Leu-W-Pro-D-Ala
wherein X is selected from Ser(Emo), hSer(Emo); Y is selected from Lys(Emo), Dab(Emo), Z is selected from D-Lys(Emo), D-Dab(Emo), D-Orn(Emo), D-Ser(Emo) D-hSer(Emo); W is selected from Lys(Emo), Dab(Emo), Orn(Emo), Ser(Emo), hSer(Emo); and pharmaceutically acceptable salts, esters, amides and hydrates thereof; or
iii) Ac-Nal-4-chloro-D-Phe-D-Pal-X-Tyr-Y-Leu-Arg-Pro-D-Ala
wherein X is selected from Ser(Emo), hSer(Emo); Y is selected from D-Cit(Emo), D-Lys(Emo), D-Dab(Emo), D-Orn(Emo), D-Ser(Emo) d-hSer(Emo); D-Nal denotes D-3-(2′-naphtyl)-alanine; D-Pal denotes 3-(3′-pyridyl)-alanine; and pharmaceutically acceptable salts, amides, esters and hydrates thereof.

74. A pharmaceutical preparation comprising as an active ingredient a GnRH analog according to claim 71, and a pharmaceutically acceptable carrier or diluent.
